(19) United States
(12) Patent Application Publication
(10) Pub. No.: US 2023/0377749 A1
(43) Pub. Date: Nov. 23, 2023

(54) SYSTEMS AND METHODS FOR ASSESSING SPEECH, LANGUAGE, AND SOCIAL SKILLS

(71) Applicants: Arizona Board of Regents on behalf of Arizona State University, Scottsdale, AZ (US); Aural Analytics, Inc., Scottsdale, AZ (US)

(72) Inventors: Visar BERISHA, Tempe, AZ (US); Julie LISS, Scottsdale, AZ (US); Rohit VOLETI, Scottsdale, AZ (US); Shira HAHN, Scottsdale, AZ (US); Gabriela STEGMANN, Scottsdale, AZ (US)

(21) Appl. No.: 18/248,349

(22) PCT Filed: Oct. 8, 2021
(86) PCT No.: PCT/US2021/054277

§ 371 Date: Apr. 7, 2023

Related U.S. Application Data
(60) Provisional application No. 63/089,482, filed on Oct. 8, 2020.

(51) Int. Cl. G16H 50/20; G16H 20/70; G10L 15/22; G10L 25/63; G10L 25/66

(52) U.S. Cl. CPC G16H 50/20; G16H 20/70; G10L 15/22; G10L 25/63; G10L 25/66; G10L 2015/225

(57) ABSTRACT

Disclosed herein are platforms, systems, software, and methods for evaluating social behavior. Speech or audio data can be analyzed to identify elemental language and acoustic components of speech that are used to determine higher order effects such as social behavior. Disclosed herein are models developed to address the assessment of mental health status (e.g. diagnosis and assessment of neurocognition and symptom ratings). In some embodiments, disclosed herein are models configured to predict performance on social and functional competency assessments. The present disclosure demonstrates the ability of a set of language features to provide several relevant upstream and/or downstream clinical assessments on audio derived data such as transcripts that were never seen during model training and showed consistent performance on all tasks of interest.

### CROSS-REFERENCE TO RELATED APPLICATIONS

This application claims the benefit of U.S. Provisional Application Ser. No. 68/089,482, filed Oct. 8, 2020, the contents of which are incorporated by reference herein in their entirety.

### BACKGROUND OF THE INVENTION

The increasing prevalence of severe mental illness and impairment caused by schizophrenia and bipolar disorder (BD) present significant challenges to individuals, families and healthcare providers all over the globe. Analysis of the Global Burden of Disease studies from 1990-2017 has shown that BD and schizophrenia impact approximately 4.53 million and 1.13 million people worldwide, respectively, with both conditions showing sharp increases in case incidences as populations continue to age over the past few decades. While prevalence of these conditions may be considered relatively low, the healthcare, social, and financial costs associated with them are disproportionately large and burdensome. The symptoms associated with these conditions are a primary source of disability for affected individuals, and this can have a drastic detrimental impact on real world functional outcomes. Those suffering from these conditions may face harmful outcomes such as severe social isolation; additionally, these impairments pose significant impacts on functional outcomes such as maintenance of employment or personal relationships. In the case of severe schizophrenia, these are also associated with premature mortality [8].

### SUMMARY OF THE INVENTION

Disclosed herein are platforms, systems, software, and methods for evaluating social behavior. Speech or audio can be analyzed to identify the elemental components of language and/or acoustics that make up speech. This information can be evaluated to assess higher level behaviors such as social skills.

There is a pressing need to address the increasing burdens imposed upon society by severe mental illness, including schizophrenia and bipolar disorder. Technological advancements in speech and natural language processing (NLP) have led to many recent studies in which automated computational models make use of speech and language features to assess and ascertaining the presence of mental illness, but there are many hurdles toward implementing their use in clinical practice. Disclosed herein are relevant and interpretable sets of language features (based on a theoretical framework) which are validated on conversational transcripts collected from a social skills assessment of individuals. The individuals may have associated mental illness, dysfunction, or impairment such as schizophrenia or bipolar disorder.

Disclosed herein are models developed to address the assessment of mental health status (e.g. diagnosis and assessment of neurocognition and symptom ratings). In some embodiments, disclosed herein are models configured to predict performance on social and functional competency assessments. The present disclosure demonstrates the ability of a set of language features to provide several relevant upstream and/or downstream clinical assessments on audio-derived data such as transcripts that were never seen during model training and showed consistent performance on all tasks of interest. The technological solution disclosed herein addresses a wide variety of clinical concerns with a limited set of language samples, which provides a means by which clinical practitioners can make use of audio data to aid in the assessment of mental health status, detection or evaluation of mental illness, and/or associated symptoms or impairments.

Disclosed herein are systems, methods, and media for evaluating social competency. Social competence or competency refers to the emotional, cognitive, and behavioral skills for functional engagement with others. In some embodiments, the systems, methods, and media disclosed herein are configured to evaluate novel, more sensitive behavioral endpoints reflective of functional competency, for example, those defined by the Specific Level of Functioning (SLOF) scale, which evaluates individuals in the domains of interpersonal relationships, participation in community and household activities, and work skills.

According to the Diagnostic and Statistical Manual of Mental Disorders (DSM-5), speech and language abnormalities are among several cardinal criteria for diagnosing schizophrenia and BD and establishing symptom severity. As a result, speech and language measures may be useful biomarkers in digital health applications. FIG. 3 shows the central role that speech and language production plays in mental health disorders. On one hand, speech and language are manifestations of the underlying upstream neurological changes the individual is experiencing. On the other hand, speech and language impairments have important downstream consequences on activities of daily living and participation (social interactions, work, etc.). The use of computational linguistic and speech processing methods to evaluate prognostic and diagnostic biomarkers for the detection of schizophrenia and BD is the upstream problem highlighted in FIG. 3.

In some aspects, disclosed herein are systems, methods, and media for using automated speech and language metrics to objectively assess downstream problems, such as the impact of disease symptoms on social and functional competency. Disclosed herein are automated speech and language features which are guided by a theoretical model of speech and language production. These features are used herein to develop robust models for addressing both the upstream and/or the downstream problems. Validation of the models is carried out by evaluating them out-of-sample on test data that the machine learning models were not trained on during development.

The instant disclosure provides at least one unified set of speech and language measures that is interpretable and can support multiple contexts of use. The utility of objective measures for earlier diagnosis and prognosis is self-evident; for example, the identification of symptoms or onset of psychosis allows for earlier and potentially more effective interventions and treatments. However, the downstream problem may be equally important, and has largely been neglected by earlier work in this field. Fundamentally, it is the end-goal of all interventions to improve social participation and quality of life. There is a concerted effort to develop digital therapeutics that target social competency in patients with schizophrenia, and objective proxies for constructs like social competency, which impact participation and quality of life, are critical in evaluating the real-world impact of pharmacological and neurobehavioral interventions.

In one aspect, disclosed herein is a computer-implemented method for automated evaluation of social competence, functional competence, or communication of an individual, said method comprising: (a) receiving, with a computing device, audio data comprising speech captured from said individual; (b) processing, with said computing device, said audio data to determine one or more elemental components comprising at least one of a language component or an acoustic component; and (c) evaluating, with said computing device, said social competence, functional competence, or communication in said individual based upon said one or more elemental components. In some embodiments, said one or more elemental components comprise said at least one language component and said at least one acoustic component. In some embodiments, said at least one language component corresponds to at least one of the following categories: verbal volition, language affect, lexical diversity, lexical density, language complexity, semantic similarity, or conversational coherence. In some embodiments, said at least one acoustic component corresponds to at least one of the following categories: articulation, prosody, phonation, resonance, or respiration. In some embodiments, further comprising capturing said speech from said individual. In some embodiments, capturing said speech from said individual comprises prompting said individual to perform a task. In some embodiments, said task is configured to assess story recall, complex picture description, category naming, object recall, affect, social skills, sentence reading, sustained phonation, diadochokinetic rate, or any combination thereof. In some embodiments, said task comprises a guided conversation between said individual and a third party. In some embodiments, said third party comprises a dialogue bot or a virtual avatar. In some embodiments, said audio data comprising said speech is actively collected during a task performed by said individual or passively collected in the absence of said task. In some embodiments, said audio data comprising said speech is actively collected from a guided conversation between said individual and a third party, actively collected from an open conversation between said individual with a third party, actively collected using a digital therapeutic, or actively collected using a dialogue bot or avatar. In some embodiments, said digital therapeutic comprises an activity, game, or video or audio recording. In some embodiments, said digital therapeutic comprises interactive content providing instructions or guidance to said individual. In some embodiments, said digital therapeutic comprises cognitive behavioral therapy. In some embodiments, said audio data comprising said speech is captured using a mobile computing device. In some embodiments, said mobile computing device is a wearable device. In some embodiments, said mobile computing device is a smart device. In some embodiments, mobile computing device comprises a tablet, a smartphone, smartwatch, smart glasses or head-mounted display, smart jewelry, or a wearable activity tracker. In some embodiments, further comprising displaying an output indicative of said social competence, functional competence, or communication of said individual evaluated based upon said one or more elemental components. In some embodiments, further comprising providing a report or summary comprising said output and instructions or recommendations based upon said social competence or communication. In some embodiments, further comprising providing a digital therapeutic for improving or managing said social competence, functional competence, or communication of said individual. In some embodiments, said output further comprises an indication of one or more social skills associated with said social competence or communication. In some embodiments, one or more of the receiving, processing, and evaluating steps are performed at least partly using cloud computing or a cloud-based server. In some embodiments, said audio data comprises one or more audio clips no more than about 10 minutes in total length. In some embodiments, further comprising providing an output comprising said evaluation of said social competence or communication of said individual through a web portal, mobile application, or remote computing device. In some embodiments, processing said audio data comprises parsing said audio data into a transcript of spoken words and associated speech acoustics. In some embodiments, processing said audio data comprises diarizing the transcript of spoken words and generating a text-acoustics alignment. In some embodiments, processing said audio data comprises estimating background noise and/or removing background noise from said audio data. In some embodiments, said audio data is evaluated using one or more first models to determine said one or more elemental components. In some embodiments, said social competence, functional competence, or communication are evaluated using one or more second models configured to receive input data comprising said one or more elemental components and generate an output indicating a performance level of said social competence or communication. In some embodiments, said one or more second models comprise an downstream model configured to determine a downstream effect on social competence, functional competence, or communication associated with a cognitive dysfunction or mental illness. In some embodiments, said one or more first models are configured to analyze features extracted from said transcript of spoken words and associated speech acoustics in order to determine said one or more elemental components. In some embodiments, further comprising evaluating said individual for a cognitive dysfunction or mental illness. In some embodiments, said cognitive dysfunction or mental illness comprises Schizophrenia, Alzheimer's, dementia, Parkinson's, autism or autism spectrum disorder, multiple sclerosis, depression, formal thought disorder, or Bipolar Disorder. In some embodiments, said evaluation for said cognitive dysfunction or mental illness comprises a presence, a severity or stage, or a risk for said cognitive dysfunction or mental illness. In some embodiments, the evaluation for said cognitive dysfunction or mental illness is performed using one or more upstream models configured to evaluate for said cognitive dysfunction or mental illness. In some embodiments, said evaluation performed using one or more downstream models generates a diagnosis or assessment of neurocognition or symptom ratings. In some embodiments, said social competence, functional competence, or communication of said individual is associated with a presence or risk of Schizophrenia, Alzheimer's, dementia, Parkinson's, autism or autism spectrum disorder, multiple sclerosis, depression, formal thought disorder, or Bipolar Disorder. In some embodiments, said evaluation of said social competence, functional competence, or communication is performed using one or more models generated according to a machine learning algorithm.

In another aspect, disclosed herein is a computing system for automated evaluation of social competence, functional competence, or communication of an individual, comprising: (a) a processor; and (b) a non-transitory computer readable storage medium encoded with executable instructions that cause the processor to: receive audio data comprising speech captured from said individual; process said audio data to determine one or more elemental components comprising at least one of a language component or an acoustic component; and evaluate said social competence, functional competence, or communication in said individual based upon said one or more elemental components. In some embodiments, said one or more elemental components comprise said at least one language component and said at least one acoustic component. In some embodiments, said at least one language component corresponds to at least one of the following categories: verbal volition, language affect, lexical diversity, lexical density, language complexity, semantic similarity, or conversational coherence. In some embodiments, said at least one acoustic component corresponds to at least one of the following categories: articulation, prosody, phonation, resonance, or respiration. In some embodiments, the processor is further caused to capture said speech from said individual. In some embodiments, capturing said speech from said individual comprises prompting said individual to perform a task. In some embodiments, said task is configured to assess story recall, complex picture description, category naming, object recall, affect, social skills, sentence reading, sustained phonation, diadochokinetic rate, or any combination thereof. In some embodiments, said task comprises a guided conversation between said individual and a third party. In some embodiments, said third party comprises a dialogue bot or a virtual avatar. In some embodiments, said audio data comprising said speech is actively collected during a task performed by said individual or passively collected in the absence of said task. In some embodiments, said audio data comprising said speech is actively collected from a guided conversation between said individual and a third party, actively collected from an open conversation between said individual with a third party, actively collected using a digital therapeutic, or actively collected using a dialogue bot or avatar. In some embodiments, said digital therapeutic comprises an activity, game, or video or audio recording. In some embodiments, said digital therapeutic comprises interactive content providing instructions or guidance to said individual. In some embodiments, said digital therapeutic comprises cognitive behavioral therapy. In some embodiments, said audio data comprising said speech is captured using a mobile computing device. In some embodiments, said mobile computing device is a wearable device. In some embodiments, said mobile computing device is a smart device. In some embodiments, said mobile computing device comprises a tablet, a smartphone, smartwatch, smart glasses or head-mounted display, smart jewelry, or a wearable activity tracker. In some embodiments, the processor is further caused to display an output indicative of said social competence, functional competence, or communication of said individual evaluated based upon said one or more elemental components. In some embodiments, the processor is further caused to provide a report or summary comprising said output and instructions or recommendations based upon said social competence or communication. In some embodiments, the processor is further caused to provide a digital therapeutic for improving or managing said social competence, functional competence, or communication of said individual. In some embodiments, said output further comprises an indication of one or more social skills associated with said social competence or communication. In some embodiments, one or more of the receive, process, and evaluate steps are performed at least partly using cloud computing or a cloud-based server. In some embodiments, said audio data comprises one or more audio clips no more than about 10 minutes in total length. In some embodiments, the processor is further caused to provide an output comprising said evaluation of said social competence or communication of said individual through a web portal, mobile application, or remote computing device. In some embodiments, processing said audio data comprises parsing said audio data into a transcript of spoken words and associated speech acoustics. In some embodiments, processing said audio data comprises diarizing the transcript of spoken words and generating a text-acoustics alignment. In some embodiments, processing said audio data comprises estimating background noise and/or removing background noise from said audio data. In some embodiments, said audio data is evaluated using one or more first models to determine said one or more elemental components. In some embodiments, said social competence, functional competence, or communication are evaluated using one or more second models configured to receive input data comprising said one or more elemental components and generate an output indicating a performance level of said social competence or communication. In some embodiments, said one or more second models comprise an downstream model configured to determine a downstream effect on social competence, functional competence, or communication associated with a cognitive dysfunction or mental illness. In some embodiments, said one or more first models are configured to analyze features extracted from said transcript of spoken words and associated speech acoustics in order to determine said one or more elemental components. In some embodiments, wherein the processor is further operative to evaluate said individual for a cognitive dysfunction or mental illness. In some embodiments, said cognitive dysfunction or mental illness comprises Schizophrenia, Alzheimer's, dementia, Parkinson's, autism or autism spectrum disorder, multiple sclerosis, depression, formal thought disorder, or Bipolar Disorder. In some embodiments, said evaluation for said cognitive dysfunction or mental illness comprises a presence, a severity or stage, or a risk for said cognitive dysfunction or mental illness. In some embodiments, the evaluation for said cognitive dysfunction or mental illness is performed using one or more upstream models configured to evaluate for said cognitive dysfunction or mental illness. In some embodiments, said evaluation performed using one or more upstream models generates a diagnosis or assessment of neurocognition or symptom ratings. In some embodiments, said social competence, functional competence, or communication of said individual is associated with a presence or risk of Schizophrenia, Alzheimer's, dementia, Parkinson's, autism or autism spectrum disorder, multiple sclerosis, depression, formal thought disorder, or Bipolar Disorder. In some embodiments, said evaluation of said social competence, functional competence, or communication is performed using one or more models generated according to a machine learning algorithm.

In another aspect, disclosed herein is a non-transitory computer readable storage medium encoded with instructions that, when executed by a processor, cause said processor to: receive audio data comprising speech captured from said individual; process said audio data to determine one or more elemental components comprising at least one of a language component or an acoustic component; and evaluate said social competence, functional competence, or communication in said individual based upon said one or more elemental components. In some embodiments, said one or more elemental components comprise said at least one language component and said at least one acoustic component. In some embodiments, said at least one language component corresponds to at least one of the following categories: verbal volition, language affect, lexical diversity, lexical density, language complexity, semantic similarity, or conversational coherence. In some embodiments, said at least one acoustic component corresponds to at least one of the following categories: articulation, prosody, phonation, resonance, or respiration. In some embodiments, the processor is further caused to capture said speech from said individual. In some embodiments, capturing said speech from said individual comprises prompting said individual to perform a task. In some embodiments, said task is configured to assess story recall, complex picture description, category naming, object recall, affect, social skills, sentence reading, sustained phonation, diadochokinetic rate, or any combination thereof. In some embodiments, said task comprises a guided conversation between said individual and a third party. In some embodiments, said third party comprises a dialogue bot or a virtual avatar. In some embodiments, said audio data comprising said speech is actively collected during a task performed by said individual or passively collected in the absence of said task. In some embodiments, said audio data comprising said speech is actively collected from a guided conversation between said individual and a third party, actively collected from an open conversation between said individual with a third party, actively collected using a digital therapeutic, or actively collected using a dialogue bot or avatar. In some embodiments, said digital therapeutic comprises an activity, game, or video or audio recording. In some embodiments, said digital therapeutic comprises interactive content providing instructions or guidance to said individual. In some embodiments, said digital therapeutic comprises cognitive behavioral therapy. In some embodiments, said audio data comprising said speech is captured using a mobile computing device. In some embodiments, said mobile computing device is a wearable device. In some embodiments, said mobile computing device is a smart device. In some embodiments, said mobile computing device comprises a tablet, a smartphone, smartwatch, smart glasses or head-mounted display, smart jewelry, or a wearable activity tracker. In some embodiments, the processor is further caused to display an output indicative of said social competence, functional competence, or communication of said individual evaluated based upon said one or more elemental components. In some embodiments, the processor is further caused to provide a report or summary comprising said output and instructions or recommendations based upon said social competence or communication. In some embodiments, the processor is further caused to provide a digital therapeutic for improving or managing said social competence, functional competence, or communication of said individual. In some embodiments, said output further comprises an indication of one or more social skills associated with said social competence or communication. In some embodiments, one or more of the receive, process, and evaluate steps are performed at least partly using cloud computing or a cloud-based server. In some embodiments, said audio data comprises one or more audio clips no more than about 10 minutes in total length. In some embodiments, the processor is further caused to provide an output comprising said evaluation of said social competence or communication of said individual through a web portal, mobile application, or remote computing device. In some embodiments, processing said audio data comprises parsing said audio data into a transcript of spoken words and associated speech acoustics. In some embodiments, processing said audio data comprises diarizing the transcript of spoken words and generating a text-acoustics alignment. In some embodiments, processing said audio data comprises estimating background noise and/or removing background noise from said audio data. In some embodiments, said audio data is evaluated using one or more first models to determine said one or more elemental components. In some embodiments, said social competence, functional competence, or communication are evaluated using one or more second models configured to receive input data comprising said one or more elemental components and generate an output indicating a performance level of said social competence or communication. In some embodiments, said one or more second models comprise an downstream model configured to determine a downstream effect on social competence, functional competence, or communication associated with a cognitive dysfunction or mental illness. In some embodiments, said one or more first models are configured to analyze features extracted from said transcript of spoken words and associated speech acoustics in order to determine said one or more elemental components. In some embodiments, wherein the processor is further operative to evaluate said individual for a cognitive dysfunction or mental illness. In some embodiments, said cognitive dysfunction or mental illness comprises Schizophrenia, Alzheimer's, dementia, Parkinson's, autism or autism spectrum disorder, multiple sclerosis, depression, formal thought disorder, or Bipolar Disorder. In some embodiments, said evaluation for said cognitive dysfunction or mental illness comprises a presence, a severity or stage, or a risk for said cognitive dysfunction or mental illness. In some embodiments, the evaluation for said cognitive dysfunction or mental illness is performed using one or more upstream models configured to evaluate for said cognitive dysfunction or mental illness. In some embodiments, said evaluation performed using one or more upstream models generates a diagnosis or assessment of neurocognition or symptom ratings. In some embodiments, said social competence, functional competence, or communication of said individual is associated with a presence or risk of Schizophrenia, Alzheimer's, dementia, Parkinson's, autism or autism spectrum disorder, multiple sclerosis, depression, formal thought disorder, or Bipolar Disorder. In some embodiments, said evaluation of said social competence, functional competence, or communication is performed using one or more models generated according to a machine learning algorithm.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative cases, in which the principles of the invention are utilized, and the accompanying drawings (also “Figure” and “FIG.” herein), of which:

FIG. 1 is a schematic diagram depicting a system for assessing parameters of speech for upstream and downstream analyses relating to social and functional competence.

FIG. 2 is a flow diagram illustrating a series of audio pre-processing steps, feature extraction, and analysis according to some embodiments of the present disclosure.

FIG. 3 shows the relationship between speech and language characteristics that are manifestations of the underlying upstream neurological changes the patient is experiencing. Speech and language abnormalities have important downstream consequences on activities of daily living and participation (e.g. social interactions, work, etc.). The upstream problem is using speech and language analysis for diagnosis or prognosis, and the downstream problem is using speech and language analysis for assessment of social and functional competency.

FIG. 4 shows a speech production block diagram model with the conceptualization, formulation, and articulation stages of spoken language production for assessing cognitive health using transcribed conversations.

FIG. 5 shows linear regression models developed to predict the downstream outcomes (a and b) and upstream cognitive and symptom ratings (c, d, and e) on the out-of-sample transcripts. The performance of these models are summarized in Tables shown in FIG. 9 and FIG. 10.

FIG. 6 shows out-of-sample logistic regression classification results for two models that were developed: (a) Clinical Participants vs. Healthy Controls, and (b) BD Participants vs. Sz/Sza Participants.

FIG. 7 shows a table with participant statistics for clinical downstream assessments of social and functional competency. Healthy control participants were only evaluated on the SSPA task.

FIG. 8 shows a table with participant statistics for clinical upstream assessments of neurocognition and symptoms. Healthy control participants were not evaluated and are excluded.

FIG. 9 shows a table with the performance of the linear regression models developed to predict performance on downstream outcomes in social and functional competency, namely in the Social Skills Performance Assessment (SSPA) and Specific Level of Functioning (SLOF) tasks. All participants were evaluated on the SSPA task (from which the transcripts originated), but only clinical participants (those with Sz/Sza or BD) were evaluated for the SLOF tasks. The table shows the performance of the models in terms of coefficient of determination (R2), the Pearson correlation coefficient (PCC), mean absolute error (MAE), and mean squared error (MSE) between the predicted and actual outcomes for each task for both the samples used for model development (cross-validation) and new unseen transcripts (out-of-sample).

FIG. 10 shows a table with the performance of the linear regression models developed to predict performance on upstream outcomes in neurocognition and symptom assessment. The table shows the performance of the models in terms of coefficient of determination (R2), the Pearson correlation coefficient (PCC), mean absolute error (MAE), and mean squared error (MSE) between the predicted and actual outcomes for each task for both the samples used for model development (cross-validation) and new unseen transcripts (out-of-sample).

FIG. 11 shows a table with the results from the two upstream logistic regression classification experiments performed on the language samples collected from the SSPA task. The first aims to differentiate between clinical (Sz/Sza or BD) participants and healthy control participants, whereas the second aims to differentiate between the Sz/Sza and BD participants. The results are reported with the confusion matrix, receiver operating characteristic area-under-curve (AUC), and a weighted average of precision, recall, and F1 score for each class prediction. Results are provided for both the cross-validation and out-of-sample participants for both experiments.

FIG. 12 shows equations for cosine similarity (1), type-to-token ratio (TTR) (2), Brune′t's Index (BI) (3), and Honore's Statistic (HS) (4).

FIG. 13 shows a table listing non-limiting examples of tasks that can be used to elicit speech for evaluating various types of assessments such as cognitive-linguistic and memory, affect, social skills, an speech motor.

FIG. 14 shows a graph plotting the relationship between volition measured via speech analysis to (a) SSPA assessment score and (b) SLOF (interpersonal+activity+work), with respect to bipolar and schizophrenia (r-values shown).

FIG. 15. shows a non-limiting example of a measurement model for assessing the language and the various features associated with language (e.g. verbal volition, semantic coherence, lexical diversity, grammatical complexity) to make predictions, for example, as to social competence, depression, cognition, functional capacity, and PANSS.

### DETAILED DESCRIPTION OF THE INVENTION

Disclosed herein are platforms, systems, software, and methods for evaluating social behavior. Input data such as speech or spoken audio can be analyzed using advanced speech and/or voice analytics to determine social skills or behavior, for example, in the context of cognitive disorders or impairments such as schizophrenia. Spoken audio can be analyzed for speech or sounds made by an individual and deconstructed to identify elemental components of language and/or acoustics that make up the speech. The elemental components of language can include volition, affect, lexical diversity, lexical density, language complexity, semantic similarity, appropriateness of response, etc. The elemental components of acoustics can include articulation, prosody (rhythm of speech), phonation, and respiration (how individual breathes during speech). These elemental components can be input into one or more algorithms that generate output indicative of one or more of depression, social competence, PANSS, cognition, and/or functional capacity. These measurements can be generated within the context of certain cognitive disorders, impairments or deficits such as schizophrenia, bipolar disorder, depression, schizoaffective, formal thought disorder, and other related disorders or conditions.

Automated language analytics is important for assessment of patients with (or at-risk for) thought disorders. As incoherent language is a common symptom across several thought disorders, most of the existing work has focused on computational models of semantically incoherent speech.

Language as a predictor of clinical condition has evaluated formal thought disorder (FTD) by comparing healthy control participants and those exhibiting FTD by using latent semantic analysis (LSA) to generate objective estimates of language similarity scores across samples elicited using a variety of tasks. The LSA has also been used to predict the onset of psychosis in young individuals deemed to be at clinical high-risk. Neural word and sentence embeddings (e.g. word2vec and GloVe) have also been used to assess similar types of coherence in speech samples from those with schizophrenia or BD. A novel approach using neural word embeddings has been proposed, in which a vector unpacking approach was used to decompose an average sentence vector into its most significant meaning components whereby low semantic density for given language elicitation tasks could serve as a reliable predictor for the onset of psychosis. Beyond semantics, other aspects of language have been computationally analyzed for individuals with schizophrenia and BD. For example, different features have been measured that are related to syntax, conversational pragmatics, several measures of language complexity, and ambiguous pronouns.

Previous work has focused on a data-driven approach to identifying language metrics as useful prognostic and diagnostic markers for schizophrenia and BD. Furthermore, the studies have focused on the utility of these metrics for solving the upstream problems of diagnosis and prognosis. However, limited investigation has been conducted to understand the relationship of these metrics to clinical symptoms or assessment of psychosis. Disclosed herein is at least one holistic set of interpretable speech and language features, guided by a theoretical model of speech production. Using this interpretable feature set, machine learning models are trained for predicting upstream and downstream variables associated with mental illness or impairment, for example, using a deeply phenotyped sample of participants with schizophrenia and bipolar disorder. The predictive abilities of the models are validated using test samples which were not used for model development.

One advantage of the present disclosure is the provisioning of systems, methods, and media that leverage the formalization of a model of speech and language production and associated interpretable computational features to assess the impairments caused by schizophrenia and BD.

Another advantage of the present disclosure is the provisioning of systems, methods, and media that utilize new models that demonstrate how these interpretable metrics can be used to: (1) assess real-world functional outcomes as measured by a variety of social and functional assessments (downstream problem) and (2) assess clinical symptoms, measures of neurocognition, and classify individuals into their diagnostic groups (upstream problem).

### Framework for Spoken Language Production

Overview

Responding to an interlocutor in a conversation is a cognitively demanding task. This is addressed using one or more models that characterize spoken language production as a complex, multi-stage event consisting of three major processes:

1. Conceptualization: involves abstract idea formation and the intent or volition to communicate the idea.

Formulation: involves selection and sequencing of words and the precise linguistic construction of an utterance, along with a sensorimotor score for muscle activation.

Articulation: involves execution of this sensorimotor score by activation and coordination of speech production musculature (e.g. respiratory, phonatory, articulatory, etc.)

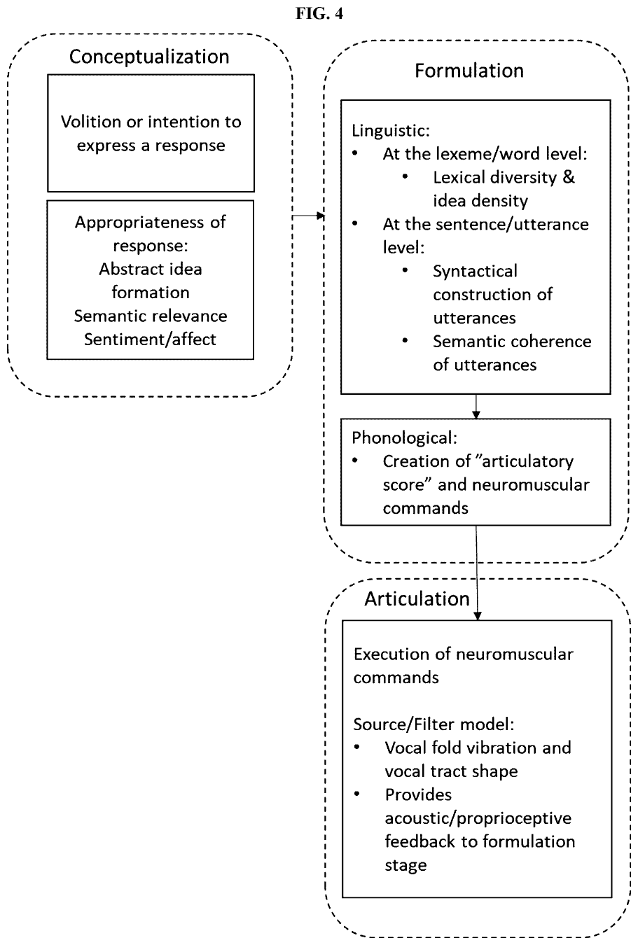

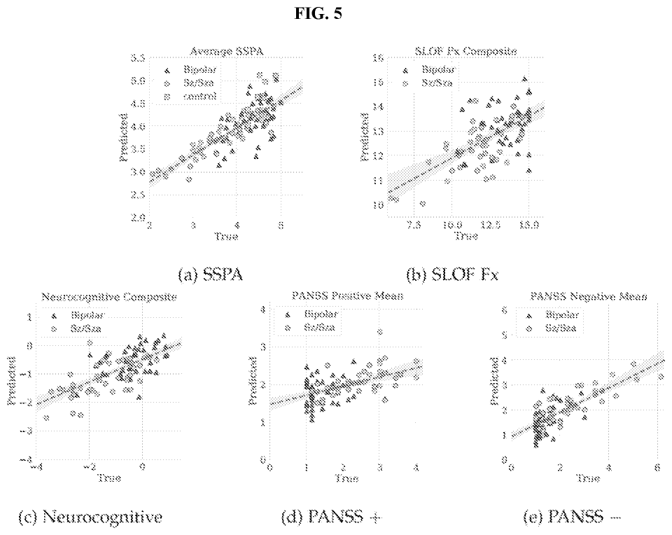

FIG. 4 illustrates this model which serves as a guide for a new representation of speech especially useful for clinical applications. The three processes can be further subdivided into a set of interpretable characteristics, each of which can be measured by a constellation of features. In some aspects, the present disclosure provides a representation of language production that (1) is sensitive to impairment by thought and mood disorders (e.g. schizophrenia and BD) and (2) can be quantified and assessed by automated computational techniques in NLP. In some embodiments, the models disclosed herein incorporate features associated with conceptualization. In some embodiments, the models disclosed herein incorporate features associated with formulation.

Conceptualization Process Features

Non-limiting examples of variables and characteristics that fall under the conceptualization process are described below. For each, provide herein is a high-level description of the features that reflect the variable or characteristic.

Volition: Refers to an individual's desire to verbally express a response, and features that reflect volition include raw word token count, mean length of sentence, and number of turns taken in a particular dialogue sequence.

Affect: Refers to an individual's mood in terms of valence (positive or negative) and arousal (high or low). A feature extracted through sentiment analysis that reflects affect is the number of positive and negative emotion words used in participant responses.

Semantic coherence: Refers to the semantic relatedness of the participant's response to the assessor's prompt. These features are computed from numerical sentence embeddings and validated similarity measures between the prompt and each response.

Appropriateness of response: Refers to the likelihood that the participant's response follows the assessor's prompt. Features are computed using deep neural network language models (e.g. BERT [29]) to predict the probability of a response given the dialogue context and assign an appropriateness score (from 1-5) for responses.

For each of these language variables and characteristics, the feature constellations are combined and reduced into a small set of features using principal components analysis.

Formulation Process Features

The features that reflect aspects of the formulation process are described below.

Lexical Diversity: Reflects the diversity in vocabulary of a participant's speech. This includes extracted features that measure the degree to which a participant's vocabulary contains unique words, e.g. type-to-token ratio (TTR).

Lexical Density: Reflects the amount of semantic content within a response. This includes features that quantify the amount of semantic content within an utterance, for example, the ratio of content words (information-dense) to function words (information-sparse).

Syntactic Complexity: Reflects the complexity of constructed sentences during speech. This includes several features that measure the complexity of sentence construction using an automated constituency-based language parser. In general, a sentence which contains more branching once parsed is thought to have a more complex syntactic construction.

In some cases, except in the case of the Affect feature domain, all features were computed by combining the transcripts from all three scenes in the Social Skills Performance Assessment (SSPA) transcript, a role-play based assessment used to measure social competence skills in three distinct scenarios. Since the emotional nature of Scenes 2 and 3 in the SSPA task were quite distinct, these features were computed independently for just scenes 2 and 3. The present disclosure recognizes that the SSPA is a non-limiting example of an assessment or task used to measure social competence skills, and alternative tasks may also be used to generate suitable audio data. For example, a variety of clinical interview tasks can be used that target one or more specific language domains.

In some cases, certain computed formulation features were not incorporated into further downstream and upstream analysis. In some cases, a formulation feature not incorporated into further analysis was related to the frequencies of different dialogue acts (informative statements, questions, directives, and commissive statements) computed using a neural network model. In some cases, a set of features computed from neural network models related to metrics for referential cohesion was not included, in which it has been shown that factors like the usage of ambiguous pronouns in speech may be a useful biomarker for detecting schizophrenia.

Language of Schizophrenia and Bipolar Disorder

Spoken language impairments in individuals with schizophrenia and bipolar disorder can be characterized in the context of the framework outlined in the previous section. Schizophrenia is a heterogeneous condition that is primarily associated with formal thought disorder (FTD), and can present with a variety of positive or negative symptoms. Positive symptoms are those in which normal functions are expressed or distorted in excess and include hallucinations, delusions, and disorganized or incoherent “word salad” speech (schizophasia). Positive symptoms associated with schizophasia are expected to impact semantic coherence and appropriateness of response in objectively measurable ways. Negative symptoms refer to those that present some type of deficiency in individuals with schizophrenia, and may include lack of motivation (avolition or amotivation), apathy, flat affect, or negative thought disorder (poverty of speech and language). In terms of the framework in FIG. 4, it is expected these negative symptoms to have a measurable negative impact on volition, affect, lexical density, lexical diversity, and syntactic complexity. Individuals can also exhibit a subset of these symptoms at varying degrees of severity.

Bipolar disorder is characterized by the fluctuation between episodes of different depressive and manic mood states. Each mood state is associated with a variety of symptoms that impact the speech and language output of that individual. Manic episodes are characterized by pressured speech, which is described as excessively rapid and difficult to understand. It is also characterized by increased verbosity and flight of ideas, or quickly jumping from topic to topic in a disorganized manner. Depressive mood states can result in exhibiting poverty of speech and language or increased pause times, similar to impairments associated with negative symptoms of schizophrenia. Therefore, within the defined framework, depressive speech will similarly primarily impact the conceptualization stage of language production, impacting our features tapping volition and affect. Manic speech can also impact the conceptualization stage, through excessive expression that may impact the appropriateness of response or semantic coherence in a given context; in the formulation stage, there may also be measurable impacts on lexical density, lexical diversity, and syntactic complexity.

In some embodiments, audio data such as audio-derived transcripts was analyzed for individuals with varying symptom severity for schizophrenia and bipolar disorder. Language features were identified that could both be associated with these particular impairments and could also be computed automatically with modern advancements in natural language processing.

The input speech data can be obtained in various ways. Speech can be elicited from the individual via a guided conversation between the patient and the clinician (Social Skills Performance Assessment), an open conversation with another person, passively-collected speech, speech collected as a part of another task (e.g. a digital therapeutic that requires the patient to produce speech), or collected via a dialog bot/avatar.

In some cases, the language is deconstructed into a set of elemental components that are known to change with various thought disorders. These disorders can include Schizophrenia, Bipolar disorder, Depression, Schizoaffective, Formal thought disorder, etc. In some cases, the platforms, systems, software, and methods disclosed herein are configured to predict social skills based on these language dimensions.

The input data can be gathered through one or more tasks or exercises, which may be guided. In some cases, the individual is instructed to engage in or act out one or more mock scenarios during which the individual will be expected or required to engage in verbal communication, which can be with a third party (which can be another person, a digital avatar, or other stand-in, or with no one). As one example, the scenario is the individual is making plans with a friend. As another example, the scenario is the individual playing the role of a tenant meeting a new neighbor. As yet another example, the scenario is the individual playing the role of the landlord in fixing an unrepaired leak. Each scenario can be short, for example, no more than 1, 2, 3, 4, 5, 6, 7, 8, 9, or 10 minutes. Speech by the individual can be captured during the scenario and parsed and analyzed using various algorithms or models to identify the elemental components of speech, which can then be evaluated to determine downstream impact, for example, social skill or behavior competence.

Speech or audio data can be processed through a series of steps. In some cases, acoustic signals are processed to identify the elemental components of language and/or acoustics. These elemental components can be parsed on a transcript, e.g. indicating the timing of the spoken language. Each elemental component may be identified from the input data using a unique model or algorithm configured to identify that specific elemental component.

In some cases, one or more algorithms are used to recognize one or more elemental components (e.g. language components, or acoustic components) from audio data captured from an individual. In some cases, the one or more recognized elemental components are used to determine social or functional competence (e.g. social skills) of the individual using the one or more algorithms. In some cases, the one or more algorithms comprise a machine learning model. In some cases, the one or more algorithms comprise a deep learning algorithm. In some cases, a level of the social skills of the individual is determined. In some cases, the recognized elemental components and/or the social or functional competence are classified into different categories using one or more classifier. The social skills may comprise one or more skills commonly used by individuals to communicate and interact with each other comprising verbal communications or non-verbal communications, for example, through language elemental components, gestures, body language and our personal appearance. In some cases, the social skills (e.g. a level of social skills) of an individual is used to determine or predict a condition of the individual such as a cognitive disorder, a thought disorder, or a mental condition. A condition may comprise schizophrenia, bipolar disorder, depression, Schizoaffective, Formal thought disorder, etc.

In some cases, determining social competence (e.g. downstream effect such as social skills) or a cognitive condition (e.g. cognitive dysfunction or mental illness) of an individual comprises determining a probability associated with the social skills or the condition in the individual. The probability may be associated with a level of the social skills displayed in an individual. The probability may associate with a level of severity of a condition in an individual. In some cases, the one or more algorithms are used to determine a probability of a level of the social skills or a level of severity of a condition in an individual based at least in part on the recognized elemental components. In some cases, one or more processors are programmed to individually or collectively execute the one or more algorithms described herein. Execution of the one or more algorithms comprising recognizing elemental components, determining social/functional/communication competence (e.g. a level of social skills, or a probability thereof), determining a condition of an individual or a probability thereof, or a combination thereof cannot be practically performed by human mind. In some cases, the platforms, systems, software, and methods provided herein are used to diagnose, predict, prevent, or monitor a condition of an individual. For example, a treatment for a condition of an individual (e.g. schizophrenia, bipolar disorder, or depression) may be monitored using the platforms, systems, software, and methods provided herein.

Disclosed herein are computational methods used to extract linguistic features in the dimensions of interest within our given framework. In some embodiments, the algorithms and models disclosed herein are directed to the linguistic conceptualization and formulation stages of language production based on speech data such as transcripts. In some embodiments, the algorithms and methods disclosed herein incorporate an acoustic assessment of articulation using audio data. For some examples, it is also important to note that each spoken utterance by participants during a task such as SSPA occurs in a conversational context, and the features explored for modeling the conceptualization and formulation stages of language production consider this context.

Conceptualization

During the conceptualization stage, an individual forms an abstract idea of what he or she intends to speak. In a conversation, this can be measured in two ways: (1) by the total verbal output (which serves as a proxy for volition or motivation to speak), and (2) measures that objectify the appropriateness of a spoken response given the context.

Volition is most simply measured by quantifying the verbal output of an individual, which in previous work has been shown to be predictive for schizophrenia, bipolar disorder, and Alzheimer's disease (AD). In some embodiments, for a given conversation, the features used included one or more of total words spoken (W), the number of participant turns (Turns/Dialogue), average number of words spoken in each turn (Tokens/Turn), the mean length of sentences (MLS), mean length of T-unit (MLT), and the mean length of clause (MLC) as a proxy for volition and motivation to speak.

Regarding affect, the Linguistic Inquiry and Word Count (LIWC) tool is used for characterizing and categorizing the lexicon of a given body of text, which includes the ability to quantify the words in a text with categories related to affective process, such as words associated with positive and negative emotions. This provides an indirect measures of the sentiment of the speaker in a conversation. For example, LIWC sentiment analysis was conducted on transcripts to give absolute counts for words spoken by each participant in the following categories: negative emotions, positive emotions, death, sadness, anger, emotional ratio (positive to negative). To simplify the analysis, in some cases, the composite features computed on Affect for tasks (e.g. scenes 2 and 3) were derived only from negative emotions, positive emotions, and the emotional ratio statistics for the transcripts of those scenes.

Regarding semantic coherence, the deficiency in an individual's ability to form semantically coherent utterances is a hallmark of formal thought disorder associated with schizophrenia and BD. One way to do this is to study the semantic relationships between the dialogue context and each spoken utterance for a given participant. In NLP, semantics are computationally modeled with word or sentence embeddings, typically a high-dimensional vector representation of a body of text. Words or phrases used in similar semantic contexts are often represented closer together as measured by their cosine similarity, given in Equation (1),

CosSim(w₁,w₂)=cos q=wT1w2/∥w1∥2∥w2∥2 (1)

where W1 and W2 are the vector representations of two bodies of text, 9 represents the angle between the two vectors, and ∥⋅∥₂ represents the Euclidean norm. Therefore, a cosine similarity can have a maximum value of 1 if the vectors are perfectly aligned, indicating identical semantics. In some cases, semantic vector representations of each utterance spoken by the assessor or participant in the SSPA task are generated. In some cases, the unweighted averages of one or more of the word2vec, smooth inverse frequency (SIF) embeddings, and sentence representations generated by the InferSent sentence encoder are considered.

The language modeling technique, Bidirectional Encoder Representations from Transformers (BERT), can improve computational performance across a variety of NLP tasks. BERT uses a transformer neural network architecture to encode text with a large pre-trained language model that can be fine-tuned for increasing performance on particular tasks. A BERT implementation can be used to encode participant responses and the dialogue context to compute similarity scores.

In some embodiments, the final reported features under this domain consist of a set of standard statistics (for example, one or more of mean, median, maximum, minimum, standard deviation, 90th percentile, and 10th percentile) computed for each conversation using the similarity scores determined by each of the above methods.

Regarding appropriateness of response, quantifiable computational features can serve to measure the degree to which a given response can be considered “appropriate” in a given dialogue context. BERT language modeling can be used in two different ways to measure appropriateness of response, using the PyTorch implementation of BERT from the transformers Python library from Huggingface:
(1) Probability of response: BERT is trained with a next sentence prediction task as one of its auxiliary objectives. The pre-trained BERT language model can be used to compute the probability of each participant response given the previous utterance by the clinical assessor.
(2) Automated response scoring: an annotated, open-source, HUMOD (human movie dialogue) dataset can be used, which consists of dialogue context-response pairs that contain both the actual responses from movie dialogue and randomly sampled responses for each context. Human annotators assigned each response a relevancy score from 1-5, resulting in a wide range of possible response scores for a given context. In some cases, fine-tuning of the pre-trained BERT model is performed by adding a regression layer on top of the pre-trained model to score each response for a given context, and then applied to the context-response pairs for each participant response in our transcripts to automatically assign a relevancy score from 1-5 to each response.

For the response probabilities, or response scores described above, a distribution of values for each conversation and summary of basic statistics for each feature can be computed for each conversation (for example, one or more of mean, median, maximum, minimum, standard deviation, 90th percentile, and 10th percentile of each distribution of values).

Dialogue act (DA) frequencies (not used in further analysis): dialogue coherence assessment can be improved by classifying each utterance as a particular dialogue act (DA). A multi-task learning approach has been used to train a neural network model for dialogue coherence assessment, for which DA classification is one of the auxiliary tasks. The model can be trained on the DailyDialog text corpus, which is annotated classifying each utterance into one of four DAs: Inform, Question, Directive, Commissive. The trained model can be used to classify each utterance as one of the four DAs for the transcripts used. The relative frequency of each DA for each conversation can be important to (1) determine if the prevalence of particular DAs is more predictive of performance in a particular scene and (2) if there are significant group differences in the types of DAs being spoken. In some cases, this may provide limited predictive value in downstream assessment of social and functional competency, and are excluded further analysis.

Formulation

Thought and mood disorders can also disrupt the formulation stage of language production, affecting an individual's choice of words and ability to form complex linguistic constructions. The computational methodologies used to quantify the impacts on language formulation can fall into two large categories, those at the lexeme/word level (e.g. lexical diversity and density) and those at the sentence and utterance level (e.g. measures from parse trees constructed from the uttered sentences).

LEXICAL DIVERSITY Lexical diversity is a measure of unique vocabulary usage. The simplest method by which this is quantified is the type-to-token ratio (TTR), given in Equation (2) as

TTR=V/N (2)

which is simply the ratio of unique words (types, V) to total words spoken (tokens, N). However, TTR tends to plateau for longer utterances and alternative methods exist to account for this length dependence. In addition to TTR, the following measures of lexical diversity which limit the length dependence may be considered:

Moving-average type-to-token ratio (MATTR) [72]: a measurement of TTR that uses a sliding window of a fixed length for a given body of text, averaged over the length of the text.

Brunét's Index (BI): defined in Equation (3) BI=N^{V\hat{ }-0.165}, in which the exponential reduces the dependence on the total length N. Lower values for BI indicate increased diversity.

Honoré's Statistic (HS) [74]: defined in Equation (4), which emphasizes the use of words that are only spoken once (V1),

HS=100 log [N/(1−V₁/V)] (4)

Lexical density is a measure of the amount of information that is packaged within an utterance. This can be quantified by the content density, or ratio of information-dense content words (e.g. nouns, verbs, adjectives, adverbs) to information-sparse function words (e.g. prepositions, conjunctions, interjections, etc.). In some cases, an algorithm such as the Stanford part-of-speech tagger is used to identify the content and function words. In some cases, the analysis of lexical density includes one or more measures that can be used as an inverse of lexical density. Measures that can be used as an inverse of lexical density include FUNC/W (ratio of function words to total words) and UH/W (ratio of interjections to total words).

Regarding syntactic complexity, measurements of this language component can be obtained by concatenating all utterances by the subject or individual (and ignoring the speech of the clinical assessor). Next, constituency-based parsing of each sentence spoken by the participant can be performed using an algorithm such as the Stanford parser tool. This allows automatic deconstruction of a sentence or phrase into its syntactic structure.

For constituency-based parsing, the parsing algorithm can be used to de-compose each sentence spoken by the participant. In some embodiments, a syntactic complexity score such as Yngve scoring is performed for each sentence. Parse tree statistics may be considered to represent this domain. Non-limiting examples include the parse tree height and Yngve depth scores (mean, total, and maximum), a measure of embedded clause usage.

Dimensionality Reduction—Principal Component Analysis (PCA)

In some aspects, the systems, methods, and media disclosed herein obtain a large set of computed features and categorize them into one or more of the seven domains as described under conceptualization and formulation sections. Such composite variables can contain interpretable information that is clinically relevant for assessing cognitive status or health including the evaluation or diagnosis of schizophrenia and BD. For one or more of the seven domains, principal component analysis (PCA) can be performed to produce a lower-dimensional representation that contains most of the information.

As an example, a raw set of computed features that spanned the seven domains is selected. In particular for the working example, a total of 43 features were chosen. The number of principal components (PCs) used to represent each domain can be chosen such that they contain at least a minimum threshold percentage variance of all variables within that domain, for example, 85% of the variance of all variables within that domain was used in the working example. In this example, the principal components obtained were 2 PCs for volition, 4 PCs for affect, 2 PCs for lexical diversity, 2 PCs for lexical density, 1 PC for syntactic complexity, 6 PCs for semantic similarity, and 4 PCs for appropriateness of response (a total of 21 features). In some embodiments, one or more of the computed features are excluded if they co-vary with the raw number of words spoken.

In some embodiments, this interpretable feature set is used to develop one or more downstream and/or upstream prediction models. The upstream models can predict measures of mental health status, for example, they may predict neurocognition variables, symptom ratings, and diagnostic group classification (e.g. schizophrenia or bipolar disorder, or a stage or severity thereof). The downstream models can measure social and functional competency outcomes from the reduced set of computed features, which are important measures of social participation that can be tracked with language analysis.

In some embodiments, the systems, methods, and media disclosed herein utilize one or more predictions algorithms or models. Such algorithms/models can include downstream and upstream prediction models. In some cases, one or more algorithms comprise classification, regression trees, ordinary least squares regression, random forest learning classification, or logistic regression. In some cases, the one or more algorithms may comprise a machine learning model. A machine learning model comprises a classifier such as a decision/classification tree (e.g. random forest (RF) or classification and regression tree (C&RT). In some cases, a combination of 2, 3, 4, 5, 6, 7, 8, 9, 10, or more machine learning models or classifiers are used. For example, the machine learning model may comprise one or more of: linear regression, logistic regression, classification and regression tree algorithm, support vector machine (SVM), naive Bayes, K-nearest neighbor, random forest algorithm, boosted algorithm such as XGBoost and LightGBM, neural network, convolutional neural network, and recurrent neural network. In some cases, the machine learning model comprises a Gradient Boosting Decision Tree (GBDT) model. The GBDT model may be used for non-linear data. The machine learning model may comprise a supervised learning algorithm, an unsupervised learning algorithm, or a semi-supervised learning algorithm. Random forest algorithms may use a large number of individual decision trees to make a classification by choosing a mode (e.g. most frequently occurring) of classes of features as determined by the individual trees. Classification and regression trees represent a computer intensive alternative to fitting classical regression models and are typically used to determine the best possible model for a categorical or continuous response of interest based upon one or more predictors. In some cases, the statistical methods or models are trained or tested using a cohort of samples (e.g. audio data or transcripts derived therefrom) from a training data set of individuals spanning the range of classifications or regression values of the desired output.

The machine learning (ML) (e.g. machine learning model, or machine learning algorithm) may be configured to accept a plurality of input variables and to produce one or more output values based on the plurality of input variables. The plurality of input variables may comprise audio data attributable to an individual or a plurality of individuals. The audio data may be captured actively during. For example, audio data may be captured during a task or exercise with a predefined goal of generating the audio data (e.g. a guided conversation between the patient and the clinician (Social Skills Performance Assessment). The audio data may be captured actively. For example, the audio data may be captured during an activity or task where the audio data produced by the individual is not the predefined goal of the activity (e.g. a digital therapeutic that requires the patient to produce speech). In some embodiments, additional data such as medical background or history, neurocognitive data, or sociodemographic data may be included or controlled for in the data analysis.

The ML may have one or more possible output values, each comprising one of a fixed number of possible values indicating social and/or functional competence, for example, social skills (e.g. a level of social competence). The output value of an ML may comprise discrete value. An ML output value may comprise one of two or more potential values. For example, an output value may be one of two values (e.g. a presence or an absence of a social skill, a 0 or 1, a positive or a negative value). The output value may indicate a classification of the social competence and/or functional competence of an individual, for example, the social skill of an individual (e.g. level of social competence, above average or below average social skills, severity of lack of social competence). The output values may comprise more than two values. For example, a presence of a condition, an absence of a condition, or an undetermined condition (e.g. presence or absence of schizophrenia or bipolar disorder). A value may indicate a severity of a condition, for example, mild lack of social skills or severe lack of social skills. Some of the output values may comprise descriptive labels. In some cases, an output may be selected from a list of values and/or ranked values (e.g. low, normal or high levels of social competence. Some descriptive labels may be mapped to numerical values, for example, by mapping “high” to 3, “normal” to 2 and “low” to 0.

The output value of an ML may comprise a continuous (or concrete) output value. An output may comprise, for example, a probability value of at least 0 and no more than 1, or a percentage between 0% to 100% (e.g. probability of a lower than average social competence). For example, an output may provide a probability that an individual has schizophrenia base at least in part on the determined social skills of the subject. The continuous output may be normalized based on a baseline value or may be un-normalized. A threshold value may be assigned to a continuous ML output values. For example, average or median social competence can be used as a threshold to classify social skills of an individual. The threshold may comprise a number between 0 to 1 or a number between 0% to 100%. There may be two or more threshold values in the output values that may indicate a higher or a lower threshold. For example, any of the quartiles in a distribution of output values may be used as the two or more threshold values. In some cases, an ML algorithm may use a threshold to generate a binary classification. For example, above a threshold or below a threshold may correspond to a binary classification of social skills (e.g. a level of social competence) of an individual. A binary classification of social skills (e.g. a level of social competence) may assign an output value of “negative” or 0 if the data indicate that the individual has social skills that is lower than the threshold (e.g. average social skills in a plurality of individuals).

The ML comprise a trained ML. A ML may be trained using a plurality of independent training datasets. Each of the independent training datasets may comprise audio data captured from an individual, or a plurality of individuals. Independent training datasets may comprise audio data captured at different time points from an individual, or a plurality of individuals. Independent training datasets may comprise audio data associated with individuals with different levels of social skills. Independent training datasets may comprise audio data associated with individuals known to have a thought disorder. For example, training datasets may comprise audio data from individuals who may have cognitive disorders or impairments such as schizophrenia (e.g. clinically diagnosed individuals). Independent training datasets may comprise audio data from healthy individuals. The healthy individuals may have varying levels of social skills from normal to very high social skills.

An ML may be trained with at most about 500, at most about 400, at most about 200, at most about 100, at most about 50, at most about 30, at most about 20, at most about 10, at most about 8, at most about 7, at most about 6, at most about 5, at most about 4, at most about 3, at most about 2, at most about 1 independent training datasets. An ML may be trained with at least 1, 2, 3, 4, 5, 6, 7, 8, 9, 10, 20, 50, 100, 200, 300, 400, 500, 600, or more independent training datasets.

The ML may be trained with a first number of independent training datasets associated with individuals known to have a thought disorder and a second number of independent training datasets associated with healthy individuals. The first number of independent training may be no more than the second number of independent training datasets. The first number of independent training datasets may be equal to the second number of independent training datasets. The first number of independent training datasets may be greater than the second number of independent training datasets. In some instances, determining an outcome (e.g. social and/or functional competence in an individual, or detection of a cognitive or mental illness or impairment such as schizophrenia or bipolar disorder) using an ML based on the elemental component of audio data has an accuracy, specificity, sensitivity, positive predictive value (PPV), a negative predictive value (NPV), or a combination thereof. In some cases, an outcome is a positive outcome. In some cases, a positive outcome is determining lack of social response in an individual. In some cases, an outcome is a negative outcome. In some cases, a negative outcome is determining normal or above normal social skills in an individual.

An accuracy of an output of a machine learning algorithm or model (e.g. an upstream or downstream prediction model) may be calculated as the percentage of outcomes that are correctly determined or classified. The outcome may be negative or positive. An ML may be configured to determine social skills (e.g. a level of social competence) of an individual based upon one or more elemental components with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%; for at least about 50, at least about 100, at least about 150, at least about 200, at least about 250, at least about 300, or more than about 300 independent datasets. For example, the accuracy can be calculated as the percentage of individuals correctly identified by ML to lack social skills.

A positive predictive value (PPV) of a ML may be calculated as the percentage of the outcomes determined as positive using the ML that are truly positive outcomes. For example, the percentage of correctly identifying an individual for having a thought disorder or lack of social skills using the ML where the individual truly has a thought disorder or lack of social skills (e.g. for example as confirmed by clinical diagnosis). A PPV may also be referred to as a precision. The ML may be configured to have a PPV of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%.

A negative predictive value (NPV) of a ML may be calculated as the percentage of the outcomes determined as negative by ML that are truly negative outcomes. For example, identifying an individual for not having a thought disorder or does not have a lack of social skills by ML that truly does not have a thought disorder or does not have a lack of social skills (e.g. for example as confirmed by clinical diagnosis). The ML may be configured to have an NVP of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%.

A sensitivity of a ML may be calculated as the percentage of truly positive outcomes correctly determined or classified as positive. For example, the percentage of individuals who truly have a thought disorder or lack of social skills (e.g. for example as confirmed by clinical diagnosis) that were determined as positive for the thought disorder or lack of social skills. The ML may be configured to have a sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%. A sensitivity may also be referred to as a recall.

A specificity of identifying of an ML may be calculated as the percentage of truly negative outcomes that were correctly determined or identified by the ML as negative outcomes. For example, the percentage of individuals who do not have a thought disorder or lack of social skills (e.g. for example as confirmed by clinical diagnosis) that were correctly determined by ML not to have the thought disorder or lack of social skills. An ML may be configured to have a specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more than about 99%.

An Area-Under-Curve (AUC) may be calculated as an integral of the Receiver Operator Characteristic (ROC) curve (e.g. the area under the ROC curve) associated with the ML in classifying or determining social skills (e.g. a level of social competence) or a thought disorder in an individual. The ML may be configured to determine social skills (e.g. a level of social competence) with an AUC of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

The ML may be adjusted or tuned to improve a performance, an accuracy, a PPV, an NPV, a sensitivity, a specificity, or an AUC of determining social skills (e.g. a level of social competence) or a thought disorder in an individual based on elemental component from an audio data of the individual. The ML may be adjusted or tuned by adjusting parameters of the ML (e.g. a set of threshold values, or weights of a neural network). The ML may be adjusted or tuned substantially continuously during the training process or after the training process has completed.

### Systems And Methods for Assessing Speech

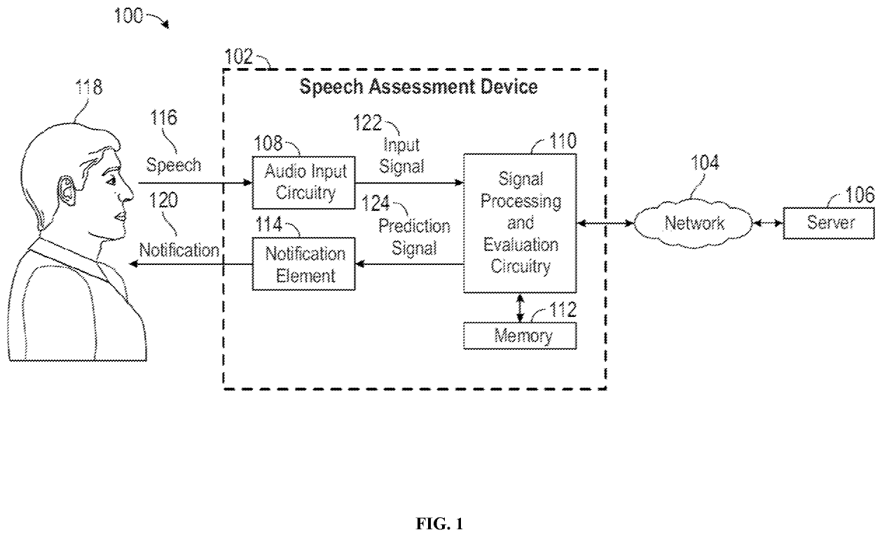

FIG. 1 is a diagram of a system 100 for assessing speech, the system comprising a speech assessment device 102, a network 104, and a server 106. The speech assessment device 102 comprises audio input circuitry 108, signal processing circuitry 110, memory 112, and at least one notification element 114. In certain embodiments, the signal processing circuitry 110 may include, but not necessarily be limited to, audio processing circuitry. In some cases, the signal processing circuitry is configured to provide at least one speech assessment signal (e.g. generated outputs based on algorithmic/model analysis of input feature measurements) based on characteristics of speech provided by a user (e.g. speech or audio stream or data). The audio input circuitry 108, notification element(s) 114, and memory 112 may be coupled with the signal processing circuitry 110 via wired connections, wireless connections, or a combination thereof. The speech assessment device 102 may further comprise a smartphone, a smartwatch, a wearable sensor, a computing device, a headset, a headband, or combinations thereof. The speech assessment device 102 may be configured to receive speech 116 from a user 118 and provide a notification 120 to the user 118 based on processing the speech 116 and any associated signals to generate output corresponding to upstream or downstream analyses using one or more trained models relating to social or functional competence. Such analyses may be associated with cognitive dysfunction or mental illness (e.g. cognitive impairment, dementia, etc.).

The audio input circuitry 108 may comprise at least one microphone. In certain embodiments, the audio input circuitry 108 may comprise a bone conduction microphone, a near field air conduction microphone array, or a combination thereof. The audio input circuitry 108 may be configured to provide an input signal 122 that is indicative of the speech 116 provided by the user 118 to the signal processing circuitry 110. The input signal 122 may be formatted as a digital signal, an analog signal, or a combination thereof. In certain embodiments, the audio input circuitry 108 may provide the input signal 122 to the signal processing circuitry 110 over a personal area network (PAN). The PAN may comprise Universal Serial Bus (USB), IEEE 1394 (FireWire) Infrared Data Association (IrDA), Bluetooth, ultra-wideband (UWB), Wi-Fi Direct, or a combination thereof. The audio input circuitry 108 may further comprise at least one analog-to-digital converter (ADC) to provide the input signal 122 in digital format.

The signal processing circuitry 110 may comprise a communication interface (not shown) coupled with the network 104 and a processor (e.g. an electrically operated microprocessor (not shown) configured to execute a pre-defined and/or a user-defined machine readable instruction set, such as may be embodied in computer software) configured to receive the input signal 122. The communication interface may comprise circuitry for coupling to the PAN, a local area network (LAN), a wide area network (WAN), or a combination thereof. The processor may be configured to receive instructions (e.g. software, which may be periodically updated) for extracting and/or computing one or more speech or acoustic features relevant to downstream analysis of social or functional competence of the user 118.

Generating an assessment or evaluation of the user 118 (e.g. social or functional competence or communication) can include measuring one or more of the speech features described herein.

Machine learning algorithms or models based on these speech measures may be used assess social or functional competence and changes thereof. In some embodiments, the output generated by the algorithms and models described herein is generated and monitored over time. Such data may be provided to a user as a report or summary, for example, a timeline of the user's social and functional competence over a certain number of years.

In certain embodiments, such machine learning algorithms (or other signal processing approaches) may analyze a panel of multiple speech features extracted from one or more speech audios using one or more algorithms or models to generate an evaluation or assessment of social or functional competence.

In certain embodiments, the processor may comprise an ADC to convert the input signal 122 to digital format. In other embodiments, the processor may be configured to receive the input signal 122 from the PAN via the communication interface. The processor may further comprise level detect circuitry, adaptive filter circuitry, voice recognition circuitry, or a combination thereof. The processor may be further configured to process the input signal 122 using one or more metrics or features derived from a speech input signal and produce a speech/audio assessment signal, and provide a prediction signal 124 to the notification element 114. The prediction signal 124 may be in a digital format, an analog format, or a combination thereof. In certain embodiments, the prediction signal 124 may comprise one or more of an audible signal, a visual signal, a vibratory signal, or another user-perceptible signal. In certain embodiments, the processor may additionally or alternatively provide the prediction signal 124 (e.g. predicted social competence score or classification according to a cognitive dysfunction or mental illness) over the network 104 via a communication interface.

The processor may be further configured to generate a record indicative of the cognitive function signal 124. The record may comprise a sample identifier and/or an audio segment indicative of the speech 116 provided by the user 118. In certain embodiments, the user 118 may be prompted to provide current symptoms or other information about their current well-being to the speech assessment device 102 for assessing language and/or acoustic elements of the audio/speech and associated social or functional competence. Such information may be included in the record, and may further be used to aid in identification or further prediction of changes in social or functional competence.

The record may further comprise a location identifier, a time stamp, a physiological sensor signal (e.g. heart rate, blood pressure, temperature, or the like), or a combination thereof being correlated to and/or contemporaneous with the speech signal 124. The location identifier may comprise a Global Positioning System (GPS) coordinate, a street address, a contact name, a point of interest, or a combination thereof. In certain embodiments, a contact name may be derived from the GPS coordinate and a contact list associated with the user 118. The point of interest may be derived from the GPS coordinate and a database including a plurality of points of interest. In certain embodiments, the location identifier may be a filtered location for maintaining the privacy of the user 118. For example, the filtered location may be “user's home”, “contact's home”, “vehicle in transit”, “restaurant”, or “user's work”. In certain embodiments, the record may include a location type, wherein the location identifier is formatted according to the location type.

The processor may be further configured to store the record in the memory 112. The memory 112 may be a non-volatile memory, a volatile memory, or a combination thereof. The memory 112 may be wired to the signal processing circuitry 110 using an address/data bus. In certain embodiments, the memory 112 may be portable memory coupled with the processor.

In certain embodiments, the processor may be further configured to send the record to the network 104, wherein the network 104 sends the record to the server 106. In certain embodiments, the processor may be further configured to append to the record a device identifier, a user identifier, or a combination thereof. The device identifier may be unique to the speech assessment device 102. The user identifier may be unique to the user 118. The device identifier and the user identifier may be useful to a medical treatment professional and/or researcher, wherein the user 118 may be a patient of the medical treatment professional.

The network 104 may comprise a PAN, a LAN, a WAN, or a combination thereof. The PAN may comprise USB, IEEE 1394 (FireWire) IrDA, Bluetooth, UWB, Wi-Fi Direct, or a combination thereof. The LAN may include Ethernet, 802.11 WLAN, or a combination thereof. The network 104 may also include the Internet.

The server 106 may comprise a personal computer (PC), a local server connected to the LAN, a remote server connected to the WAN, or a combination thereof. In certain embodiments, the server 106 may be a software-based virtualized server running on a plurality of servers.

In certain embodiments, at least some signal processing tasks may be performed via one or more remote devices (e.g. the server 106) over the network 104 instead of within a speech assessment device 102 that houses the audio input circuitry 108.

In certain embodiments, a speech assessment device 102 may be embodied in a mobile application configured to run on a mobile computing device (e.g. smartphone, smartwatch) or other computing device. With a mobile application, speech samples can be collected remotely from patients and analyzed without requiring patients to visit a clinic. A user 118 may be periodically queried (e.g. two, three, four, five, or more times per day) to provide a speech sample, for example, by actively requesting performance of a task. For example, the notification element 114 may be used to prompt the user 118 to provide speech/audio 116 from which the input signal 122 is derived, such as through a display message or an audio alert. The notification element 114 may further provide instructions to the user 118 for providing the speech 116 (e.g. displaying a passage for the user 118 to read). In certain embodiments, the notification element 114 may request current symptoms or other information about the current well-being of the user 118 to provide additional data for analyzing the speech 116. Alternatively, or in combination, speech/audio can be passively collected through a user's phone as they go about their day.

In certain embodiments, a notification element may include a display (e.g. LCD display) that displays text and prompts the user to read the text. Each time the user provides a new sample using the mobile application, one or more features (e.g. elemental components of the speech/audio such as language components and acoustic components) of the user's speech abilities may be automatically extracted and/or computed. For example, certain audio features may require further algorithmic computation to calculate a feature useful for determining social or functional competence such as appropriateness of response which may be computed using deep neural network language models such as BERT which can predict the probability of a response given the dialogue context and assign an appropriateness score for responses. One or more machine-learning algorithms based on these metrics or features may be implemented to aid in identifying and/or predicting social or functional competence of the user that is associated with the speech capabilities.

In certain embodiments, a user may download a mobile application to a personal computing device (e.g. smartphone), optionally sign into the application, and follow the prompts on a display screen. Once recording has finished, the audio data may be automatically uploaded to a secure server (e.g. a cloud server or a traditional server) where the signal processing and machine learning algorithms operate on the recordings.

### Audio Processing

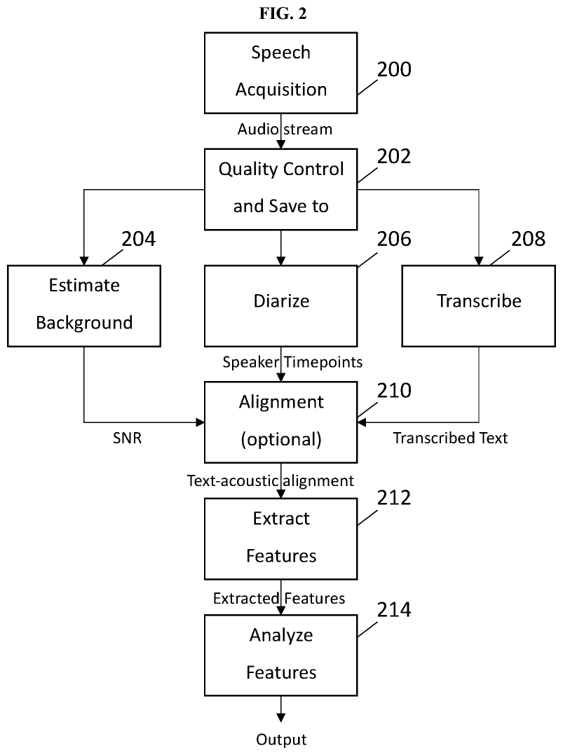

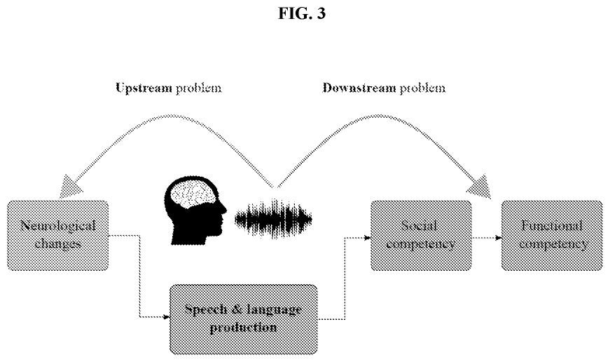

As shown in FIG. 2, the process for speech/language feature extraction and analysis can include one or more steps such as speech acquisition 200, quality control 202, background noise estimation 204, diarization 206, transcription 208, optional alignment 210, feature extraction 212, and/or feature analysis 214. In some embodiments, the systems, devices, and methods disclosed herein include a speech acquisition step. Speech acquisition 200 can be performed using any number of audio collection devices. Examples include microphones or audio input devices on a laptop or desktop computer, a portable computing device such as a tablet, mobile devices (e.g. smartphones), digital voice recorders, audiovisual recording devices (e.g. video camera), and other suitable devices. In some cases, these devices are configured with software to provide digital therapeutics, for example, cognitive behavioral therapy. In some embodiments, the speech or audio is acquired through passive collection techniques. For example, a device may be passively collecting background speech via a microphone without actively eliciting the speech from a user or individual. The device or software application implemented on the device may be configured to begin passive collection upon detection of background speech. Alternatively, or in combination, speech acquisition can include active elicitation of speech. For example, a mobile application implemented on the device may include instructions prompting speech by a user or individual. In some embodiments, the user is prompted to provide conversational responses to questions or a verbal description such as, for example, a picture description. In some embodiments, the systems, devices, and methods disclosed herein utilize a dialog bot or chat bot that is configured to engage the user or individual in order to elicit speech. As an illustrative example, the bot may engage in a conversation with the user (e.g. via a graphic user interface such as a smartphone touchscreen or via an audio dialogue). Alternatively or in combination with a conversation, the bot may simply provide instructions to the user to perform a particular task (e.g. instructions to vocalize pre-written speech or sounds). In some cases, the speech or audio is not limited to spoken words, but can include nonverbal audio vocalizations made by the user or individual. For example, the user may be prompted with instructions to make a sound that is not a word for a certain duration.

In some embodiments, the systems, devices, and methods disclosed herein include a quality control step 202. The quality control step may include an evaluation or quality control checkpoint of the speech or audio quality. Quality constraints may be applied to speech or audio samples to determine whether they pass the quality control checkpoint. Examples of quality constraints include (but are not limited to) signal to noise ratio (SNR), speech content (e.g. whether the content of the speech matches up to a task the user was instructed to perform), audio signal quality suitability for downstream processing tasks (e.g. speech recognition, diarization, etc.). Speech or audio data that fails this quality control assessment may be rejected, and the user asked to repeat or redo an instructed task (or alternatively, continue passive collection of audio/speech). Speech or audio data that passes the quality control assessment or checkpoint may be saved on the local device (e.g. user smartphone, tablet, or computer) and/or on the cloud. In some cases, the data is both saved locally and backed up on the cloud. In some embodiments, one or more of the audio processing and/or analysis steps are performed locally or remotely on the cloud.

In some embodiments, the systems, devices, and methods disclosed herein include background noise estimation 204. Background noise estimation can include metrics such as a signal-to-noise ratio (SNR). SNR is a comparison of the amount of signal to the amount background noise, for example, ratio of the signal power to the noise power in decibels. Various algorithms can be used to determine SNR or background noise with non-limiting examples including data-aimed maximum-likelihood (ML) signal-to-noise ratio (SNR) estimation algorithm (DAML), decision-directed ML SNR estimation algorithm (DDML) and an iterative ML SNR estimation algorithm.

In some embodiments, the systems, devices, and methods disclosed herein perform audio analysis of speech/audio data stream such as speech diarization 206 and speech transcription 208. The diarization process can include speech segmentation, classification, and clustering. In some cases when there is only one speaker, diarization is optional. The speech or audio analysis can be performed using speech recognition and/or speaker diarization algorithms. Speaker diarization is the process of segmenting or partitioning the audio stream based on the speaker's identity. As an example, this process can be especially important when multiple speakers are engaged in a conversation that is passively picked up by a suitable audio detection/recording device. In some embodiments, the diarization algorithm detects changes in the audio (e.g. acoustic spectrum) to determine changes in the speaker, and/or identifies the specific speakers during the conversation. An algorithm may be configured to detect the change in speaker, which can rely on various features corresponding to acoustic differences between individuals. The speaker change detection algorithm may partition the speech/audio stream into segments. These partitioned segments may then be analyzed using a model configured to map segments to the appropriate speaker. The model can be a machine learning model such as a deep learning neural network. Once the segments have been mapped (e.g. mapping to an embedding vector), clustering can be performed on the segments so that they are grouped together with the appropriate speaker(s).

Techniques for diarization include using a Gaussian mixture model, which can enable modeling of individual speakers that allows frames of the audio to be assigned (e.g. using Hidden Markov Model). The audio can be clustered using various approaches. In some embodiments, the algorithm partitions or segments the full audio content into successive clusters and progressively attempts to combine the redundant clusters until eventually the combined cluster corresponds to a particular speaker. In some embodiments, algorithm begins with a single cluster of all the audio data and repeatedly attempts to split the cluster until the number of clusters that has been generated is equivalent to the number of individual speakers. Machine learning approaches are applicable to diarization such as neural network modeling. In some embodiments, a recurrent neural network transducer (RNN-T) is used to provide enhanced performance when integrating both acoustic and linguistic cues. Examples of diarization algorithms are publicly available (e.g. Google).

Speech recognition (e.g. transcription of the audio/speech) may be performed sequentially or together with the diarization. The speech transcript and diarization can be combined to generate an alignment of the speech to the acoustics (and/or speaker identity). In some cases, passive and active speech are evaluated using different algorithms. Standard algorithms that are publicly available and/or open source may be used for passive speech diarization and speech recognition (e.g. Google and Amazon open source algorithms may be used). Non-algorithmic approaches can include manual diarization. In some embodiments, diarization and transcription are not required for certain tasks. For example, the user or individual may be instructed or required to perform certain tasks such as sentence reading tasks or sustained phonation tasks in which the user is supposed to read a pre-drafted sentence(s) or to maintain a sound for an extended period of time. In such tasks, transcription may not be required because the user is being instructed on what to say. Alternatively, certain actively acquired audio may be analyzed using standard (e.g. non-customized) algorithms or, in some cases, customized algorithms to perform diarization and/or transcription. In some embodiments, the dialogue or chat bot is configured with algorithm(s) to automatically perform diarization and/or speech transcription while interacting with the user

In some embodiments, the speech or audio analysis comprises alignment 210 of the diarization and transcription outputs. The performance of this alignment step may depend on the downstream features that need to be extracted. For example, certain features require the alignment to allow for successful extraction (e.g. features based on speaker identity and what the speaker said), while others do not. In some embodiments, the alignment step comprises using the diarization output to extract the speech from the speaker of interest. Standard algorithms may be used with non-limiting examples including Kaldi, gentle, Montreal forced aligner), or customized alignment algorithms (e.g. using algorithms trained with proprietary data).

In some embodiments, the systems, devices, and methods disclosed herein perform feature extraction 212 from one or more of the SNR, diarization, and transcription outputs. One or more extracted features can be analyzed 214 to predict or determine an output comprising one or more composites or related indicators of speech production. In some embodiments, the output comprises an indicator of a physiological condition such as a cognitive status or impairment (e.g. dementia-related cognitive decline).

The systems, devices, and methods disclosed herein may implement or utilize a plurality or chain or sequence of models or algorithms for performing analysis of the features extracted from a speech or audio signal. In some embodiments, the plurality of models comprises multiple models individually configured to generate specific composites or perceptual dimensions. In some embodiments, one or more outputs of one or more models serve as input for one or more next models in a sequence or chain of models. In some embodiments, one or more features and/or one or more composites are evaluated together to generate an output. In some embodiments, a machine learning algorithm or ML-trained model (or other algorithm) is used to analyze a plurality of feature or feature measurements/metrics extracted from the speech or audio signal to generate an output such as a composite. In some embodiments, the systems, devices, and methods disclosed herein combine the features to produce one or more composites that describe or correspond to an outcome, estimation, or prediction, for example, corresponding to social or functional competence or communication.

### Computer Systems

The present disclosure provides computer systems that are programmed to implement methods of the disclosure. In some cases, the computer system can be configured to collect audio data, speech produced by an individual, a conversation between an individual and another person (e.g. with a therapist, or with a dialog bot/avatar), process the data, identify an elemental component in the audio date, determining or evaluating social skills of an individual based on the elemental component. The computer system can regulate various aspects of the present disclosure, such as, for example, storing data in short term memory or long term memory, processing the data, frequency of processing data, tuning an ML to improve a performance, an accuracy, a PPV, an NPV, a sensitivity, a specificity, or an AUC of an outcome of the ML. The computer system can be an electronic device of a user or a computer system that is remotely located with respect to the electronic device. The electronic device can be a mobile electronic device.

The computer system includes a central processing unit (CPU, also “processor” and “computer processor” herein), which can be a single core or multi core processor, or a plurality of processors for parallel processing. The computer system also includes memory or memory location (e.g. random-access memory, read-only memory, flash memory), electronic storage unit (e.g. hard disk), communication interface (e.g. network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g. user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g. a mobile device, a digital camera, or an audio recording device). Examples of remote computer systems include personal computers (e.g. portable PC), slate or tablet PC's (e.g. Apple® iPad, Samsung® Galaxy Tab), telephones, Smart phones (e.g. Apple® iPhone, Android-enabled device, Blackberry®), or personal digital assistants. The user can access the computer system via the network.

Methods as described herein can be implemented by way of machine (e.g. computer processor) executable code stored on an electronic storage location of the computer system 501, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g. read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, an outcome of an ML (e.g. a level of social skill or social competence of an individual). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, collect audio data, speech produced by an individual, a conversation between an individual and another person (e.g. with a therapist, or with a dialog bot/avatar), process the data, identify an elemental component in the audio date, determining or evaluating social skills of an individual based on the elemental component.

### Computer Program

In some cases, the computer system comprises a computer program. One feature of the computer program includes a sequence of instructions, executable in the digital processing device's CPU (e.g. by CPU), written to perform a specified task. In some cases, computer readable instructions are implemented as program modules, such as functions, features, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions are combined or distributed as desired in various environments. In some instances, a computer program comprises one sequence of instructions or a plurality of sequences of instructions. A computer program may be provided from one location. A computer program may be provided from a plurality of locations. In some cases, a computer program includes one or more software modules. In some cases, a computer program includes, in part or in whole, one or more web applications, one or more mobile applications, one or more standalone applications, one or more web browser plug-ins, extensions, add-ins, or add-ons, or combinations thereof.

### Web Application

In some cases, a computer program includes a web application. In light of the disclosure provided herein, those of skill in the art will recognize that a web application may utilize one or more software frameworks and one or more database systems. A web application, for example, is created upon a software framework such as Microsoft® .NET or Ruby on Rails (RoR). A web application, in some instances, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In some cases, a web application is written in one or more markup languages, presentation definition languages, client-side scripting languages, server-side coding languages, database query languages, or combinations thereof. In some cases, a web application is written to some extent in a markup language such as Hypertext Markup Language (HTML), Extensible Hypertext Markup Language (XHTML), or eXtensible Markup Language (XML). In some cases, a web application is written to some extent in a presentation definition language such as Cascading Style Sheets (CSS). In some cases, a web application is written to some extent in a client-side scripting language such as Asynchronous Javascript and XML (AJAX), Flash® Actionscript, Javascript, or Silverlight®. In some cases, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some cases, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some instances, a computer program includes a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non-limiting examples, C, C++, C #, Objective-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some cases, a computer program includes a standalone application, which is a program that may be run as an independent computer process, not an add-on to an existing process, e.g. not a plug-in. Those of skill in the art will recognize that standalone applications are sometimes compiled. In some instances, a compiler is a computer program(s) that transforms source code written in a programming language into binary feature code such as assembly language or machine code. Suitable compiled programming languages include, by way of non-limiting examples, C, C++, Objective-C, COBOL, Delphi, Eiffel, Java™, Lisp, Python™ Visual Basic, and VB .NET, or combinations thereof. Compilation may be often performed, at least in part, to create an executable program. In some instances, a computer program includes one or more executable complied applications.

### Web Browser Plug-In

A computer program, in some aspects, includes a web browser plug-in. In computing, a plug-in, in some instances, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

In some cases, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in some instances, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Module

The medium, method, and system disclosed herein frequently comprise one or more software, servers, and database modules, or use of the same. In view of the disclosure provided herein, software modules may be created by techniques known to those of skill in the art using machines, software, and languages known to the art. The software modules disclosed herein may be implemented in a multitude of ways. In some cases, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### EXAMPLES

### Example 1

### Methods

Data Used for Model Development and Model Evaluation

Data from a total of 281 participants with a clinical diagnosis of either schizophrenia/schizoaffective (Sz/Sza) disorder (n=140) or bipolar disorder (BD) (n=141) and 22 healthy controls were used in this study. The data came from a larger study . . . .

Every participant was subject to extensive clinical evaluations that consisted of neurocognitive batteries, symptom ratings, social, and functional assessments.

LANGUAGE SAMPLES: Language samples from each participant were elicited via the Social Skills Performance Assessment (SSPA) task. The SSPA is a simple-to-administer role-playing test that can serve as a measurement of skills related to social competence. Participants in the study are asked to act out the following three “scenes” with a clinical assessor:

Scene 1 (practice): the participant plans a weekend activity with a friend (˜1 minute)

Scene 2 (scored): the participant introduces a new neighbor to his or her neighborhood (˜3 minutes)

Scene 3 (scored): the participant negotiates with a difficult landlord to fix a leak in his or her apartment (˜3 minutes)

Scenes 2 and 3 are individually scored on a scale from 1 to 5 across a variety of dimensions, such as overall interest/disinterest, affect, negotiation ability, fluency, etc. An overall score for each scene can be computed by averaging the scores across each dimension of interest for each scene.

The SSPA was administered by trained researchers at the Psychology department at Queen's University at Kingston, Ontario, Canada. The samples were manually transcribed.

DEVELOPMENT/TEST SPLIT: The data was randomly split into two sets, a development set and a test set. A table of descriptive statistics for all relevant outcome measures is shown in Tables 1 and 2. The development set was used to develop the model. Importantly, the test set was not utilized at any point during model development. Once the model was fixed, its performance was evaluated using the test set. A summary of the training and test set samples are shown in Tables 1 and 2.

Feature Dimensionality Reduction and Model Development

The large set of computed features was categorized into the seven processes described under conceptualization and formulation order to determine the composite variables that contain interpretable information that is clinically relevant for assessing cognitive or mental health status such as for individuals having or at risk for developing schizophrenia and BD. For each of the seven processes, principal component analysis (PCA) was applied to produce a lower-dimensional representation that contains most of the information.

In this example, the analysis began with a raw set of 43 computed features that spanned the seven domains. The number of principal components (PCs) used to represent each domain was chosen such that they contain at least 85% of the variance of all variables within that process. As a result, the principal components used included 2 PCs for volition, 4 PCs for affect, 2 PCs for lexical diversity, 2 PCs for lexical density, 1 PC for syntactic complexity, 6 PCs for semantic similarity, and 4 PCs for appropriateness of response (a total of 21 features). These were used along with the raw count of word tokens (W) for model development. Recent analysis has shown that many computational measures for assessment in psychosis are highly correlated simply with the number of words spoken. For this reason, we control for the raw count of word tokens in each of the models and assess the additional value provided by the more complex language measures that we computed.

This interpretable feature set was used to develop several downstream and upstream prediction models: The downstream models are:

Prediction of average SSPA score for each participant

Prediction of SLOE scores (3 subscales and overall functional competency)

The upstream models are:

Prediction of the neurocognitive composite score

Prediction of symptom ratings on the PANSS scale (positive symptoms mean and negative symptoms mean)

Diagnostic group classification models.

The details of each model and the results are provided below.

Downstream Assessment of Social and Functional Competency

For all predictive analyses, the regression models were developed and optimized using leave-one-out cross-validation (LOOCV) on only the training samples; the best performing model was selected and fixed and then subsequently evaluated on the test samples. All models were built using the features described in the above section. The total number of words spoken in each model was controlled for, as several of the features co-varied with it.

For this illustrative and non-limiting example, healthy control target scores are only available for the SSPA prediction model. All other analyses only considered the Sz/Sza and BD samples.

Social Skills Performance Assessment Score Prediction

Regression models for predicting SSPA scores using the reduced representation of the feature domains were developed and evaluated. Descriptive statistics for SSPA scores are shown in the table in FIG. 7.

A summary of the cross-validation (for model development) and out-of-sample test performance for SLOF prediction models is shown in the bottom half of the table in FIG. 9.

Specific Level of Functioning Score Prediction

The SLOF assessment for all clinical participants (Sz/Sza and BD) was independently modeled for the three different subscales: (1) interpersonal relations, (2) participation in home and community activities, and (3) work skills.

The three subscale scores are also summed into a composite functional competency score (Fx composite). Descriptive statistics for SLOF scores are shown in the table in FIG. 7.

A summary of the cross-validation (for model development) and out-of-sample test performance for SLOF prediction models is shown in the bottom half of the table in FIG. 9.

Upstream Assessment of Mental Health Status

The upstream analysis consists of three different models. The language feature domain representations were used to predict the neurocognitive composite score (a combination of several cognitive assessments) and the average values of the positive and negative symptom scale ratings (PANSS positive and negative averages). Additionally, the reduced feature sets were used to classify between the clinical group and healthy Controls and to classify the clinical group into the corresponding diagnostic groups (Sz/Sza, BD).

Neurocognitive Composite Score Prediction

Schizophrenia, schizoaffective disorder, and bipolar disorder are known to negatively impact neurocognition to varying degrees for afflicted individuals. A composite neurocognitive score was computed and reported for each clinical participant (excluding healthy control participants). In summary, the composite score consists of eight well-known neurocognitive batteries: the Rey Auditory Verbal Learning Test, Trail Making Test, letter-number span test from the Weschler Adult Intelligence Scale (WAIS), Wisconsin Card Sorting Test, Digit-Symbol Coding test from the WAIS, a semantic fluency test, d′ from the Continuous Performance Test-Identical Pairs Version, 4-digit condition, and the reading subtest of the Wide-Range Achievement Test, 3rd edition. Standardized z-scores from these tests were used to compute a composite score.

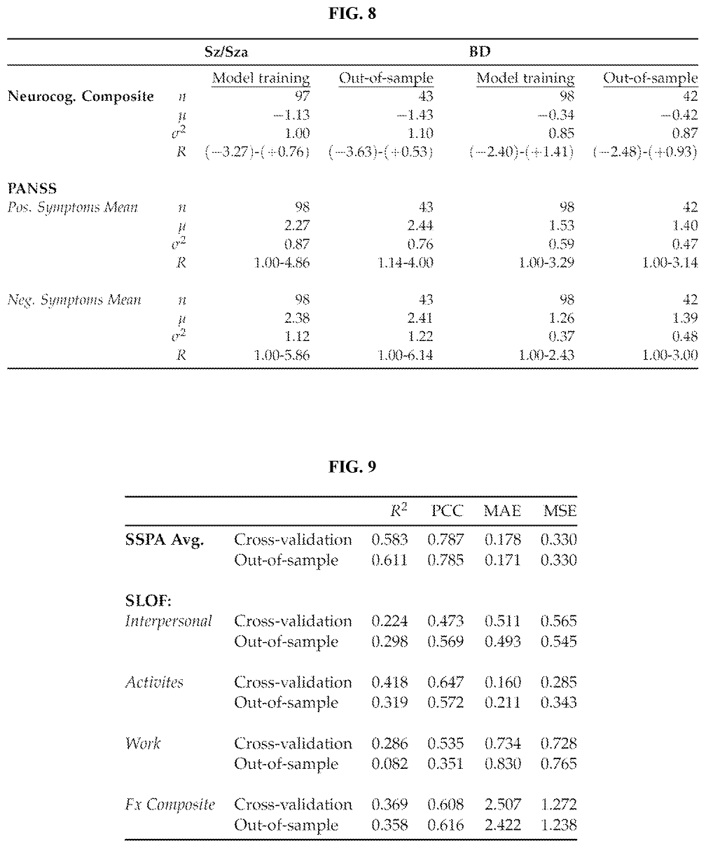

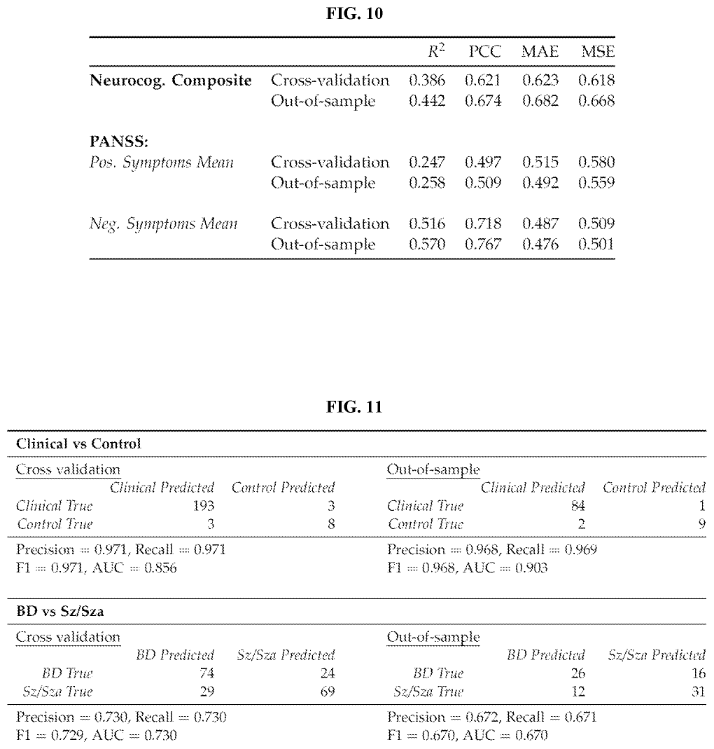

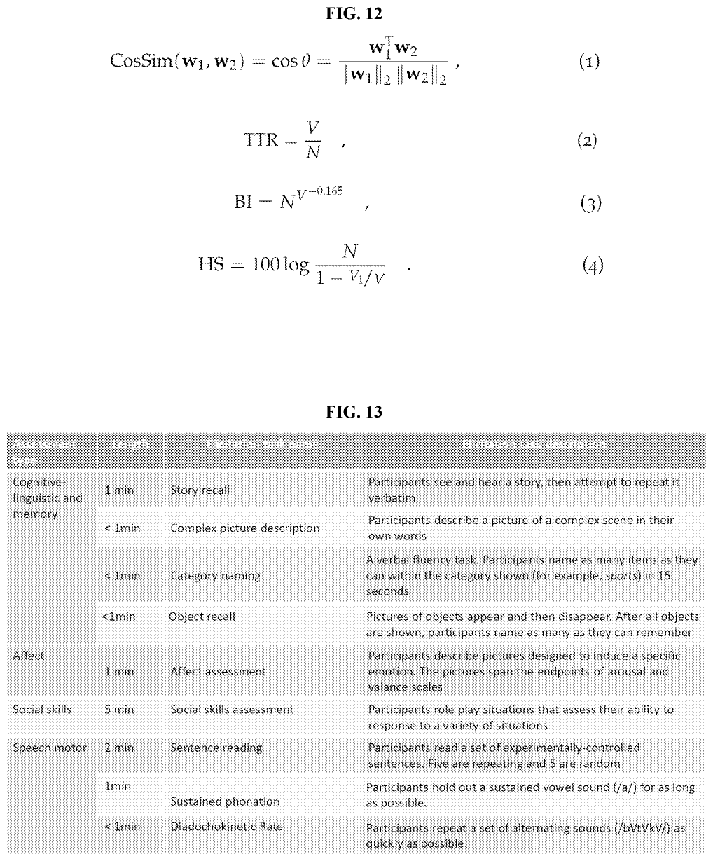

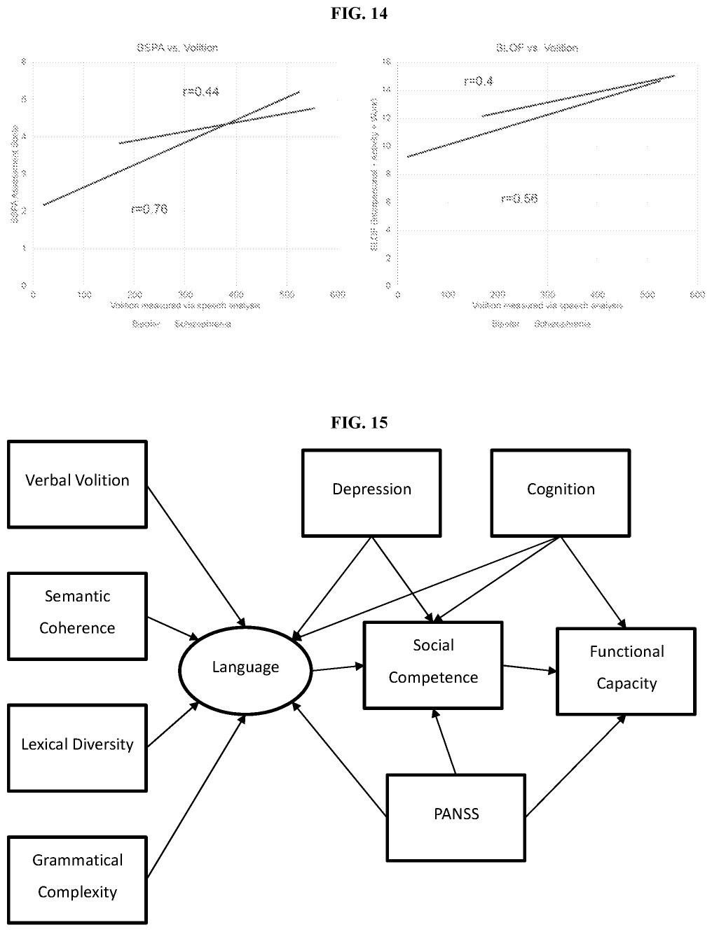

The distribution of neurocognitive composite scores are summarized in the table in FIG. 8. The performance of the regression models used to predict the composite neurocognitive score is shown in the table in FIG. 10.

Positive and Negative Symptoms Scale (PANSS) Rating Predictions

The Positive and Negative Symptoms Scale (PANSS) assessment consists of seven items that measure the severity of positive symptoms and seven items that measure the severity of negative symptoms. The average value of the positive symptom values and negative symptom values for each participant was used. The distribution across our participant groups for these Positive Symptoms Mean and Negative Symptoms Mean values are shown in the table in FIG. 8. Once again, healthy controls are excluded.

The performance of the linear regression predictive model on the training sample and out-of-sample participants is summarized as well in the table in FIG. 10.

Diagnostic Group Class Prediction

The next set of experiments in the upstream analysis aimed to correctly identify the diagnostic group to which each participant belongs using the computed language features. This was accomplished by fitting logistic regression binary classifiers on the training set of participant transcripts in two separate experiments.

The first task (Clinical vs. Control) was to identify if participants fall into a clinical diagnostic group (Sz/Sza or BD) or are a healthy control. Since there is a large discrepancy in the overall number of clinical participant transcripts (n=195 in the training data set) when compared to healthy controls (n=11 in the training data set), the SMOTE data augmentation technique was employed to generate synthetic control samples and to over-sample the minority class during cross-validation to counter the imbalance.

The next task (BD vs Sz/Sza) aimed to fit a similar logistic regression binary classifier to differentiate between the individuals that belonged to each of the two groups within the clinical transcript samples. Since the Sz/Sza and BD classes are quite balanced in support, no data augmentation or over-sampling was used.

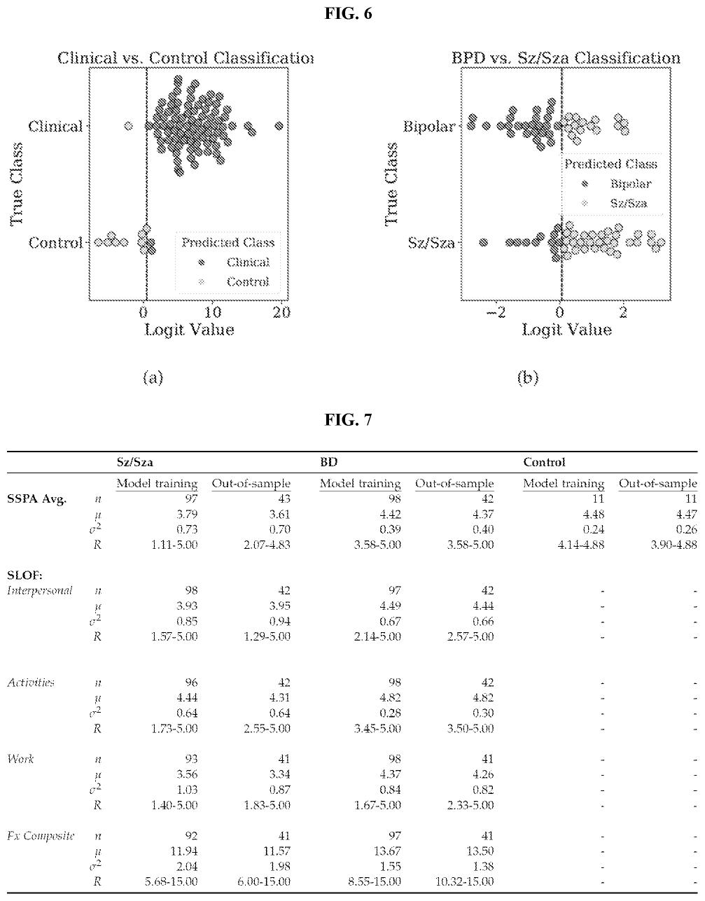

The results from both classification experiments are summarized for the cross-validation and out-of-sample participants in the table in FIG. 11. FIG. 6 shows a visual representation of both classification experiments on the out-of-sample transcripts that were not used in developing each model; it is clear in part (a) of FIG. 6 that the clinical and healthy control classification model performs very well even on new unseen transcripts, but that the Sz/Sza vs. BD classification in part (b) of FIG. 6 is more difficult. Still, from the results in the table in FIG. 11, it is clear that both models perform similarly well on the training samples and the new samples. The results are shown with weighted averages for precision, recall, and F1 score for correctly predicting each class (weighted by the support of that class). Also shown is the area-under-curve (AUC) for the receiver operating characteristic (ROC) curve to evaluate the performance of the classifier.

### Discussion

Disclosed herein are new computational models of language production and their application to evaluation of upstream clinical variables (e.g. neurocognition, symptoms, and diagnosis) and downstream clinical variables (e.g. social and functional competency).

The models were developed using a subset of the PCA representation of the feature domains. Additionally, since many computed features can co-vary with the length of the dialogue, the total number of words spoken by each participant (W) was used in each model as a normalizing mechanism. The feature domains measure varying degrees of language impairment in participants from different groups.

Volition

While the raw word count (W) was used in all our models, the principal components associated with other measures of volition were analyzed to interpret what additional value they provide for our predictive models. It was found that the PCs associated with volition were particularly useful in assessment of PANSS positive symptoms. Positive symptoms are associated with overactive expression (e.g. hallucinations, delusions) and would therefore be directly impacted by increased or decreased volition. The impact on social competency outcomes can be mediated by positive symptom severity, which is consistent with the present finding that the principal components associated with volition were also useful in predicting SSPA score outcomes.

Affect

While schizophrenia is primarily considered a thought disorder and BD is primarily considered a mood disorder, both are known to significantly impact overall affect and emotional processing. Individuals with schizophrenia often exhibit flat or “blunted” affect, a common negative symptom. In BD, individuals experience a wide range of emotions and moods. During a manic episode they may be unusually upbeat and even exhibit euphoria, and during a depressive episode they may express extreme sadness, hopelessness, worthlessness, or guilt. All of these emotional expressions are in contrast to what is expected with healthy individuals.

The computational variables for measuring emotion used the Linguistic Inquiry and Word Count (LIWC) to count the number of words that are associated with positive emotions, negative emotions, and the ratio of positive to negative emotions according to the tool's built-in dictionary of words. Additionally, the SSPA task itself is not ideal for a natural expression of emotions, as the participants are required to perform a specific exercise in which they are role-playing for a short amount of time. Still, it was surprising and unexpected to see differences in emotional processing for individuals in each group just based on these measures, as the two scored scenes (new neighbor and landlord conversations) are intended to contain very different emotional content.

Emotional processing is often thought of separately from cognitive ability, but some have considered them to be more directly linked in both BD and schizophrenia. In BD, neurocognitive and emotional deficits are known to have impacts on downstream social and functional outcomes and are closely linked. For schizophrenia, cognition may play a critical role in the maintenance of emotional information, and it is thought that neurocognitive deficits are partially responsible for the emotional disassociation that affected individuals exhibit. To this end, we found that affect played an important role in computing the neurocognitive composite score for the upstream regression model. Similarly, the downstream impacts of affect are also apparent in the model predicting overall SSPA score.

Semantic Coherence & Appropriateness of Response

Semantically incoherent speech is observed as a common occurrence for many individuals with schizophrenia (associated with Formal Thought Disorder), and it is occasionally observed for those in the manic phase of BD. Disorganized and incoherent speech has been cited as an early predictor of an oncoming psychotic episode and as a useful diagnostic feature for classifying between healthy controls and those with psychosis. In this example, the language samples that were collected for the SSPA task are conversational in nature; in particular, a goal was to accurately measure if the responses that participants give make sense with a given conversational context. This required a closer evaluation of both the semantic coherence and appropriateness of response feature domains.

When computing the variables, semantic coherence was defined as a cosine similarity score between sentence embeddings computed between participant utterances and the interviewer prompts. The appropriateness of response was considered separately as a way to score responses to a given context on an appropriateness scale, which does not necessarily take the semantics of the actual utterance into account. While these two feature domains are measured with different computational methods and approaches, they are designed to measure the same construct; that is, the goal was in understanding if a particular participant utterance is relevant given the context, and both the appropriateness of response and semantic coherence feature domains are able to provide insight on this concept. Therefore, both of these feature categories were considered together in the analysis of what they measure for the upstream and downstream outcomes.

Schizophrenia may be primarily a disorder of social communication. Those with deficits have difficulty perceiving intention and forming responses in social situations. This manifests as a core symptom of thought disorders and is known to severely negatively impact social cognition and functional outcome. For these reasons, these feature domains are both useful in the analysis of conversational transcripts in determining if a response makes sense.

In the upstream models, the average of PANSS positive symptoms, e.g. those resulting from disorganized and incoherent speech, can be predicted using features from the appropriateness domain; however, PANSS negative symptoms cannot. This is expected as the appropriateness domain includes features that assign response probability and appropriateness scores to each participant utterance in a given dialogue. As such, they can only be computed when a response is provided. By design, these features do not measure reduced volition or verbal output.

Since both of these variables are computed on conversational data, they were expected to be significant components of the models focused on clinical diagnosis. Indeed, the models revealed that features from the appropriateness and semantic coherence domains were useful in separating impaired individuals (Sz/Sza or BD) from healthy control participants. It was also found that semantic coherence in particular was significant in discriminating between individuals with Sz/Sza and BD. Positive symptom severity is also known to differ between those with each condition, which is evident from our previous observation that positive symptom severity can be predicted with these variables.

For the downstream models, appropriateness was important for predicting the overall average SSPA and SLOF Activities scores; semantic coherence variables were significant in the prediction of the other two SLOF subscales (work skills and interpersonal relationships) as well as the overall SLOF functional score (SLOF Fx). In the case of the SSPA score prediction, appropriateness of response measures were arguably among the most important features used in the regression model. Adaptive and social competency measures (such as those measured by the SSPA) are good predictors of downstream functional assessments measured by the SLOF scale. Therefore, these features were evaluated for their role in determining functional competency outcomes in our models predicting the SLOF subscale assessments. Bowie et al. found that the interpersonal relationships and work skills SLOF subscales showed direct correlation with social competency measures from the SSPA scale (C. R. Bowie et al., “Prediction of real-world functional disability in chronic mental disorders: A comparison of schizophrenia and bipolar disorder,” American Journal of Psychiatry, vol. 167, no. 9, pp. 1116-1124, 2010). They also used a separate set of adaptive competency measures and showed their strong relationship to the SLOF activities subscale; the adaptive competency test consists of the UCSD Performance-based Skills Assessment (UPSA-B) that evaluates several functional skills in communication and financial literacy. They found that there was no clear relationship between the SSPA and the activities subscale for SLOF. However, in our work, we found that our measures of appropriateness of response in a social context were important components of the SLOF activities subscale prediction. Previous work found a strong negative correlation between PANSS positive symptoms and performance on the UPSA-B evaluation. Since appropriateness was useful in measuring the severity of positive symptoms, we posit that variables from this domain serve as a proxy for positive symptom severity in predicting SLOF-Activities outcomes.

Lexical Diversity

Some declines in lexical diversity (e.g. unique vocabulary usage) are observed at late stages of aging, but are more significantly impacted when cognitive deficits are present. Lexical diversity has been found a direct indicator of a decline in cognitive ability for those with dementia, chronic traumatic encephalopathy (CTE), and Sz/Sza and BD. The present disclosure confirmed the importance of lexical diversity as the variables associated with this domain were a critical component of our upstream model predicting the neurocognitive composite score. Neurocognitive measures were found to be directly correlated with lexical diversity variables.

The neurocognitive deficits that are measured by these variables have a known impact on downstream outcomes. As with appropriateness of response and semantic coherence, there was a significant correlation between lexical diversity measures and downstream SSPA task performance. This impact on the social competency outcomes is again mediated by the positive symptom severity measured by the PANSS scale, which is also correlated with lexical diversity in our models.

Omitted Feature Domains

The final two feature domains, lexical density and syntactic complexity were not subsequently chosen to provide value for any of our upstream or downstream predictions for this particular non-limiting example. Both were evaluated due to their ability to measure important outcomes for individuals with cognitive and thought disorders. However, these particular feature domains were not found to be as significant when the transcripts were conversational and elicited short responses. However, alternative data such as transcripts derived from passively or actively captured audio of longer utterances or speech by an individual allows for these features to provide predictive utility.

Lexical density is defined as a measure of “information packaging” in a given utterance. Such measures can be useful in assessing cognitive deficits associated with mild cognitive impairment (MCI), dementia, primary progressive aphasia (PPA), and several others. These measures may be useful in evaluating upstream and downstream outcomes in cognitive and mental status or illnesses such as Sz/Sza and BD. In some cases, the conversational nature of transcripts that allow short responses may limit the utility of these variables. For example, participant responses that are quite short in nature (e.g. “yes” or “okay”) do not lend themselves well to measures of lexical density, which are more insightful with increased verbal output. Accordingly, in some embodiments, the tasks or activities utilized by the systems, methods, and media disclosed herein include tasks or activities configured to elicit or prompt a longer participant response to provide improved utility of lexical density measures. In some cases, the task or activity is configured to require a minimum length for a response (e.g. a minimum 10 word response). For example, an illustrative task or activity may require a description of an image or video, an explanation of an idea, or a summary of a story. In some cases, the response or speech (e.g. captured audio or transcript) is filtered to select for an appropriate length prior to analysis by one or more algorithms or models. In some embodiments, a prediction algorithm/model is configured to incorporate lexical density in making upstream and/or downstream predictions.

Similarly, measures of syntactic complexity are less useful when participant responses are short. Accordingly, the language sample (e.g. response to a task or activity) can be narrative in nature and contain more complete sentences, which improves the predictive utility of syntactic complexity. In some cases, the language sample is more narrative and not conversational in nature.

In some aspects, the systems, methods, and media disclosed herein utilize automated computational models developed with language samples, which can provide tremendous potential for aiding clinicians aiming to assist those with severe forms of mental illness such as schizophrenia and bipolar disorder. In some embodiments, features are selected according to a theoretical framework for language production, for example, starting with the phases of conceptualization and formulation of what one intends to say. In some embodiments, various NLP techniques are used to compute one or more features that fall within each of these domains. These feature domains can include areas of language which are known to be impaired in various cognitive or mental illnesses such as schizophrenia and bipolar disorder. Basing feature selection around this theoretical framework ensures that there is a clinical relevance to the language metrics that are being computed.

The collected samples can be utilized to perform a specific downstream task (e.g. the SSPA task), as well as allowing reasonable predictions of upstream measures of mental health status and several downstream measures of social and functional competency. In some cases, model performance was best when it came to the regression models that predicted average SSPA performance, which was expected since the SSPA transcripts were the source of our language samples. However, reasonable predictive value was unexpectedly shown for measures of neurocognition, symptom ratings, and functional competency tasks as well, as shown in the example. Additionally, classification of individuals into their respective diagnostic groups is also possible from the SSPA transcripts, even though the SSPA is not intended as a clinical diagnostic tool. Importantly, on every regression and classification model that was developed, the model performance generalized well to transcripts that were never seen during their training by an independent biostatistician. This ensures confidence that these analyses are feasible and repeatable given adequate language samples.

While preferred cases of the present invention have been shown and described herein, it will be obvious to those skilled in the art that such cases are provided by way of example only. Numerous variations, changes, and substitutions will now occur to those skilled in the art without departing from the invention. It should be understood that various alternatives to the cases of the invention described herein may be employed in practicing the invention. It is intended that the following claims define the scope of the invention and that methods and structures within the scope of these claims and their equivalents be covered thereby.

1. A computer-implemented method for automated evaluation of social competence, functional competence, or communication of an individual, said method comprising:
(a) receiving, with a computing device, audio data comprising speech captured from said individual;
(b) processing, with said computing device, said audio data to determine one or more elemental components comprising at least one of a language component or an acoustic component; and
(c) evaluating, with said computing device, said social competence, functional competence, or communication in said individual based upon said one or more elemental components.

2. The method of claim 1, wherein said one or more elemental components comprise said at least one language component and said at least one acoustic component.

3. The method of claim 1, wherein said at least one language component corresponds to at least one of the following categories: verbal volition, language affect, lexical diversity, lexical density, language complexity, semantic similarity, or conversational coherence.

4. The method of claim 1, wherein said at least one acoustic component corresponds to at least one of the following categories: articulation, prosody, phonation, resonance, or respiration.

5. The method of claim 1, further comprising capturing said speech from said individual.

6. The method of claim 5, wherein capturing said speech from said individual comprises prompting said individual to perform a task.

7. The method of claim 6, wherein said task is configured to assess story recall, complex picture description, category naming, object recall, affect, social skills, sentence reading, sustained phonation, diadochokinetic rate, or any combination thereof.

8. The method of claim 6, wherein said task comprises a guided conversation between said individual and a third party.

9. The method of claim 8, wherein said third party comprises a dialogue bot or a virtual avatar.

10. The method of claim 1, wherein said audio data comprising said speech is actively collected during a task performed by said individual or passively collected in the absence of said task.

11. The method of claim 10, wherein said audio data comprising said speech is actively collected from a guided conversation between said individual and a third party, actively collected from an open conversation between said individual with a third party, actively collected using a digital therapeutic, or actively collected using a dialogue bot or avatar.

12. The method of claim 11, wherein said digital therapeutic comprises an activity, game, or video or audio recording.

13. The method of claim 11, wherein said digital therapeutic comprises interactive content providing instructions or guidance to said individual.

14. The method of claim 11, wherein said digital therapeutic comprises cognitive behavioral therapy.

15. The method of claim 1, wherein said audio data comprising said speech is captured using a mobile computing device.

16. The method of claim 15, wherein said mobile computing device is a wearable device.

17. The method of claim 15, wherein said mobile computing device is a smart device.

18. The method of claim 15, wherein said mobile computing device comprises a tablet, a smartphone, smartwatch, smart glasses or head-mounted display, smart jewelry, or a wearable activity tracker.

19. The method of claim 1, further comprising displaying an output indicative of said social competence, functional competence, or communication of said individual evaluated based upon said one or more elemental components.

20. The method of claim 19, further comprising providing a report or summary comprising said output and instructions or recommendations based upon said social competence or communication.

21. The method of claim 19, further comprising providing a digital therapeutic for improving or managing said social competence, functional competence, or communication of said individual.

22. The method of claim 19, wherein said output further comprises an indication of one or more social skills associated with said social competence or communication.

23. The method of claim 1, wherein one or more of the receiving, processing, and evaluating steps are performed at least partly using cloud computing or a cloud-based server.

24. The method of claim 1, wherein said audio data comprises one or more audio clips no more than about 10 minutes in total length.

25. The method of claim 1, further comprising providing an output comprising said evaluation of said social competence or communication of said individual through a web portal, mobile application, or remote computing device.

26. The method of claim 1, wherein processing said audio data comprises parsing said audio data into a transcript of spoken words and associated speech acoustics.

27. The method of claim 26, wherein processing said audio data comprises diarizing the transcript of spoken words and generating a text-acoustics alignment.

28. The method of claim 26, wherein processing said audio data comprises estimating background noise and/or removing background noise from said audio data.

29. The method of claim 1, wherein said audio data is evaluated using one or more first models to determine said one or more elemental components.

30. The method of claim 29, wherein said social competence, functional competence, or communication are evaluated using one or more second models configured to receive input data comprising said one or more elemental components and generate an output indicating a performance level of said social competence or communication.

31. The method of claim 30, wherein said one or more second models comprise an downstream model configured to determine a downstream effect on social competence, functional competence, or communication associated with a cognitive dysfunction or mental illness.

32. The method of claim 29, wherein said one or more first models are configured to analyze features extracted from said transcript of spoken words and associated speech acoustics in order to determine said one or more elemental components.

33. The method of claim 1, further comprising evaluating said individual for a cognitive dysfunction or mental illness.

34. The method of claim 33, wherein said cognitive dysfunction or mental illness comprises Schizophrenia, Alzheimer's, dementia, Parkinson's, autism or autism spectrum disorder, multiple sclerosis, depression, formal thought disorder, or Bipolar Disorder.

35. The method of claim 33, wherein said evaluation for said cognitive dysfunction or mental illness comprises a presence, a severity or stage, or a risk for said cognitive dysfunction or mental illness.

36. The method of claim 33, wherein the evaluation for said cognitive dysfunction or mental illness is performed using one or more upstream models configured to evaluate for said cognitive dysfunction or mental illness.

37. The method of claim 36, wherein said evaluation performed using one or more downstream models generates a diagnosis or assessment of neurocognition or symptom ratings.

38. The method of claim 1, wherein said social competence, functional competence, or communication of said individual is associated with a presence or risk of Schizophrenia, Alzheimer's, dementia, Parkinson's, autism or autism spectrum disorder, multiple sclerosis, depression, formal thought disorder, or Bipolar Disorder.

39. The method of claim 1, wherein said evaluation of said social competence, functional competence, or communication is performed using one or more models generated according to a machine learning algorithm.

40. A computing system for automated evaluation of social competence, functional competence, or communication of an individual, comprising:
(a) a processor; and
(b) a non-transitory computer readable storage medium encoded with executable instructions that cause the processor to:
receive audio data comprising speech captured from said individual;
process said audio data to determine one or more elemental components comprising at least one of a language component or an acoustic component; and
evaluate said social competence, functional competence, or communication in said individual based upon said one or more elemental components.

41. The system of claim 40, wherein said one or more elemental components comprise said at least one language component and said at least one acoustic component.

42. The system of claim 40, wherein said at least one language component corresponds to at least one of the following categories: verbal volition, language affect, lexical diversity, lexical density, language complexity, semantic similarity, or conversational coherence.

43. The system of claim 40, wherein said at least one acoustic component corresponds to at least one of the following categories: articulation, prosody, phonation, resonance, or respiration.

44. The system of claim 40, wherein the processor is further caused to capture said speech from said individual.

45. The system of claim 44, wherein capturing said speech from said individual comprises prompting said individual to perform a task.

46. The system of claim 45, wherein said task is configured to assess story recall, complex picture description, category naming, object recall, affect, social skills, sentence reading, sustained phonation, diadochokinetic rate, or any combination thereof.

47. The system of claim 45, wherein said task comprises a guided conversation between said individual and a third party.

48. The system of claim 47, wherein said third party comprises a dialogue bot or a virtual avatar.

49. The system of claim 40, wherein said audio data comprising said speech is actively collected during a task performed by said individual or passively collected in the absence of said task.

50. The system of claim 49, wherein said audio data comprising said speech is actively collected from a guided conversation between said individual and a third party, actively collected from an open conversation between said individual with a third party, actively collected using a digital therapeutic, or actively collected using a dialogue bot or avatar.

51. The system of claim 50, wherein said digital therapeutic comprises an activity, game, or video or audio recording.

52. The system of claim 50, wherein said digital therapeutic comprises interactive content providing instructions or guidance to said individual.

53. The system of claim 50, wherein said digital therapeutic comprises cognitive behavioral therapy.

54. The system of claim 40, wherein said audio data comprising said speech is captured using a mobile computing device.

55. The system of claim 54, wherein said mobile computing device is a wearable device.

56. The system of claim 54, wherein said mobile computing device is a smart device.

57. The system of claim 54, wherein said mobile computing device comprises a tablet, a smartphone, smartwatch, smart glasses or head-mounted display, smart jewelry, or a wearable activity tracker.

58. The system of claim 40, wherein the processor is further caused to display an output indicative of said social competence, functional competence, or communication of said individual evaluated based upon said one or more elemental components.

59. The system of claim 58, wherein the processor is further caused to provide a report or summary comprising said output and instructions or recommendations based upon said social competence or communication.

60. The system of claim 58, wherein the processor is further caused to provide a digital therapeutic for improving or managing said social competence, functional competence, or communication of said individual.

61. The system of claim 58, wherein said output further comprises an indication of one or more social skills associated with said social competence or communication.

62. The system of claim 40, wherein one or more of the receive, process, and evaluate steps are performed at least partly using cloud computing or a cloud-based server.

63. The system of claim 40, wherein said audio data comprises one or more audio clips no more than about 10 minutes in total length.

64. The system of claim 40, wherein the processor is further caused to provide an output comprising said evaluation of said social competence or communication of said individual through a web portal, mobile application, or remote computing device.

65. The system of claim 40, wherein processing said audio data comprises parsing said audio data into a transcript of spoken words and associated speech acoustics.

66. The system of claim 65, wherein processing said audio data comprises diarizing the transcript of spoken words and generating a text-acoustics alignment.

67. The system of claim 65, wherein processing said audio data comprises estimating background noise and/or removing background noise from said audio data.

68. The system of claim 40, wherein said audio data is evaluated using one or more first models to determine said one or more elemental components.

69. The system of claim 68, wherein said social competence, functional competence, or communication are evaluated using one or more second models configured to receive input data comprising said one or more elemental components and generate an output indicating a performance level of said social competence or communication.

70. The system of claim 69, wherein said one or more second models comprise an downstream model configured to determine a downstream effect on social competence, functional competence, or communication associated with a cognitive dysfunction or mental illness.

71. The system of claim 68, wherein said one or more first models are configured to analyze features extracted from said transcript of spoken words and associated speech acoustics in order to determine said one or more elemental components.

72. The system of claim 40, wherein the processor is further caused to evaluate said individual for a cognitive dysfunction or mental illness.

73. The system of claim 72, wherein said cognitive dysfunction or mental illness comprises Schizophrenia, Alzheimer's, dementia, Parkinson's, autism or autism spectrum disorder, multiple sclerosis, depression, formal thought disorder, or Bipolar Disorder.

74. The system of claim 72, wherein said evaluation for said cognitive dysfunction or mental illness comprises a presence, a severity or stage, or a risk for said cognitive dysfunction or mental illness.

75. The system of claim 72, wherein the evaluation for said cognitive dysfunction or mental illness is performed using one or more upstream models configured to evaluate for said cognitive dysfunction or mental illness.

76. The system of claim 75, wherein said evaluation performed using one or more upstream models generates a diagnosis or assessment of neurocognition or symptom ratings.

77. The system of claim 40, wherein said social competence, functional competence, or communication of said individual is associated with a presence or risk of Schizophrenia, Alzheimer's, dementia, Parkinson's, autism or autism spectrum disorder, multiple sclerosis, depression, formal thought disorder, or Bipolar Disorder.

78. The system of claim 40, wherein said evaluation of said social competence, functional competence, or communication is performed using one or more models generated according to a machine learning algorithm.
